(19) United States
(12) Patent Application Publication
(10) Pub. No.: US 2015/0335790 A1
(43) Pub. Date: Nov. 26, 2015

(54) COMPOSITIONS AND METHODS FOR TEMPLATING THREE-DIMENSIONAL MINERALIZATION

(71) Applicant: UNIVERSITY OF MASSACHUSETTS MEDICAL SCHOOL, Boston, MA (US)

(72) Inventors: Jie Song, Shrewsbury, MA (US); Pingsheng Liu, Worcester, MA (US)

(21) Appl. No.: 14/652,636

(22) PCT Filed: Dec. 13, 2013
(86) PCT No.: PCT/US13/74950

§ 371 Date: Jun. 16, 2015

Related U.S. Application Data
(60) Provisional application No. 61/738,844, filed on Dec. 18, 2012.

(51) Int. Cl. A61L 27/46; A61L 27/58; A61L 27/38

(52) U.S. Cl. CPC A61L 27/46; A61L 27/3821; A61L 27/58; A61L 2430/02

(57) ABSTRACT

The invention provides novel compositions and methods for three-dimensional mineralization templated by synthetic scaffolds having zwitterionic mediators. The invention enables 3-D mineralization nucleation and growth of minerals in a well-controlled and defined manner. The composite materials prepared by the disclosed methods are cytocompatible and/or biodegradable and are suitable for use as medical implants in a variety of applications in skeletal tissue repair and regeneration. For example, cytocompatible zwitterionic sulfobetaine ligands are employed to facilitate 3-D mineralization of HA across covalently crosslinked hydrogels. The overall charge-neutral zwitterionic hydrogel effectively recruited oppositely charged precursor ions while overcame excessive swelling exhibited by anionic and cationic hydrogels under physiological conditions, resulting in denser and structurally well-integrated mineralized composite materials.

### PRIORITY CLAIMS AND RELATED PATENT APPLICATIONS

This application claims the benefit of priority from U.S. Provisional Application Ser. No. 61/738,844, filed on Dec. 18, 2012, the entire content of which is incorporated herein by reference in its entirety.

### GOVERNMENT RIGHTS

This invention was made with Government support under grant no. R01AR055615 awarded by the NIH and under grant nos. S10RR027082 and S10RR021043 awarded by the National Center for Research Resources. The Government has certain rights in the invention.

### TECHNICAL FIELDS OF THE INVENTION

The invention generally relates to compositions and methods for templated mineralization. More particularly, the invention relates to compositions and methods for three-dimensional mineralization templated by synthetic scaffolds having zwitterionic mediators, and cytocompatible and/or biodegradable composite materials, implants and uses thereof.

### BACKGROUND OF THE INVENTION

Templated 3-dimensional (3-D) mineralization in scaffolds with hydroxyapatite (HA) is of significant value for bone and dental tissue repair and regeneration. Many synthetic biomaterials designed for template-driven mineralization utilized anionic residues, cationic residues or uncharged polar ligands including in amphiphilic forms to drive the heterogeneous nucleation. (Palmer, et al. 2008 Chem Rev 108:4754-83; Hartgerink 2001 Science 294:1684-8; Cha, et al. 2011 Biomaterials 32:2695-703; Chao, et al. 2012 J Mater Chem 22, 6080-6087; Phadke, et al. 2010 Biomacromolecules 11:2060-8; Murphy, et al. 2002 J Am Chem Soc 124:1910-7; Stephenson, et al. 2008 Science 322:724-7; Taubert, et al. 2010 Macromol Biosci 10:1084-92; Koumoto, et al. 2004 Biomaterials 25:3915-21; Song, et al. 2003 J Am Chem Soc 125:1236-43; Hutchens, et al. 2006 Biomaterials 27:4661-70; Ma 2008 Adv Drug Deliver Rev 60:184-98; Chen, et al. 2011 J Am Chem Soc 133:5214-7.)

The template mineralization of such scaffolds, however, were not accomplished in a truly 3-D fashion, with either heterogeneous nucleation/growth predominantly occurring on the surface of dense substrate or homogeneous precipitation loosely trapped within macropores or porous scaffolds. In vitro mineralization of many bulk scaffolds often led to limited heterogeneous nucleation/growth of HA beyond the surface of dense substrates or homogeneous precipitation loosely trapped within porous scaffold.

It remains a major challenge to achieve tunable and well-integrated mineralization of bulk synthetic scaffold in a truly 3-D fashion. 3-D template nucleation and growth of HA has yet to be accomplished. An un-met need remains for novel methods and cytocompatible composite materials through controlled and truly three-dimensional mineralization.

### SUMMARY OF THE INVENTION

The invention provides novel compositions and methods for three-dimensional mineralization templated by synthetic scaffolds having zwitterionic mediators. The invention enables 3-D nucleation and growth of minerals in a well-controlled and defined manner. The composite materials prepared by the disclosed methods are cytocompatible and/or biodegradable and are suitable for use as medical implants in a variety of applications in skeletal and dental tissue repair and regeneration.

The invention involves the strategic utilization of zwitterionic ligands to simultaneously recruit anionic and cationic precursor ions. Both positively and negatively charged residues play pivotal roles in recruiting precursor ions or ion clusters, and lowering interfacial energy in natural biomineralization process. For example, cytocompatible zwitterionic sulfobetaine ligands are employed to facilitate 3-D mineralization of HA across covalently crosslinked hydrogels. The overall charge-neutral zwitterionic hydrogel effectively recruited oppositely charged precursor ions while overcame excessive swelling exhibited by anionic and cationic hydrogels under physiological conditions, resulting in denser and structurally well-integrated mineralized composite materials.

The methods disclosed herein allow designs and fine controls over the size, content, and spatial distribution of the mineral domains within the zwitterionic hydrogel, for example, through facile adjustments of hydrogel crosslinking densities and the supersaturation rate governing heterogeneous mineral nucleation and growth.

In one aspect, the invention generally relates to a structurally well-integrated composite material comprising 3-dimensioanl network of a synthetic polymer that comprises a zwitterionic moiety and structurally integrated mineral components throughout the 3-D network. In certain preferred embodiments, the composite material is cytocompatible. In certain preferred embodiments, the composite material is biodegradable.

In another aspect, the invention generally relates to a method for mediating 3-D mineralization. The method includes (1) providing a mixture that includes: (a) a 3-D scaffold of a polymer network, wherein the polymer network comprises a a zwitterionic moiety; (b) a mineral precursor; and (c) a pH mediator; (2) causing the pH mediator to gradually adjust the pH level of the mixture; and (3) transforming the mineral precursor into mineral deposits as templated by the 3-D scaffold.

In yet another aspect, the invention generally relates to an implant having a composite material that is characterized by a 3-D scaffold of a polymer network substantially mineralized therein hydroxyapatite crystals with direct structural integration between the polymer template and the minerals. The polymer includes a zwitterionic moiety.

In yet another aspect, the invention generally relates to a 3-dimensional scaffold that includes a 3-dimensioanl polymer network. The polymer network includes a zwitterionic moiety. In certain preferred embodiments, the 3-D polymer network is crosslinked, forming a 3-D crosslinked polymer network, such as a crosslinked hydrogel of polymethacrylate.

### BRIEF DESCRIPTION OF THE FIGURES

FIG. 1. Schematic illustrations of (a) the swelling behavior of a crosslinked 3-D zwitterionic polymer scaffold in water vs. in aqueous saline solution, and (b) 3-D HA-mineralization of a zwitterionic polymer scaffold driven by a gradual increase of supersaturation resulting from the thermal decomposition of urea.

FIG. 2. (a) Micro-CT sagittal cross-section views and (b) mineral content (n=3, determined by μ-CT) of chemically crosslinked polymethacrylate hydrogels with varied side chain chemistries and ionic charges upon mineralization. All hydrogels were mineralized at a heating rate of 0.2 ° C./min. The mineral content was normalized against equilibrium volume (EV) of the respective hydrogels in PBS. (c) Swelling ratio of the un-mineralized hydrogels (n=3) by weight (S_{w}) in pure water and PBS, respectively. (d) Temporal pH changes of the mineralization solution (n=3). (e) Zeta potential of linear pSBMA (Mₙ=12424, PDI=1.09, 3 mg/mL in 0.015 M NaCl aqueous solution) as a function of pH.

FIG. 3. (a) SEM and TEM micrographs illustrating FIB processing and thinning of a mineral noduled formed within the interior of a mineralized pSBMA specimen. (b) TEM micrograph and the corresponding (c) SAED pattern & lattice plane intensities of the FIB-thinned mineralized specimen. (d) HR-TEM micrograph and SAED (inset) of a randomly chosen area shown in b). (e) Representative EDS spectrum acquired on the ultra-thin FIB specimen.

FIG. 4. Confocal Z-stack (80 μm) images of live (green)/dead (red) rat bone marrow stromal cells (rMSC, 10⁶ cells/mL) encapsulated in the cationic (pTMAEMA), anionic (pSPMA), and zwitterionic (pSBMA) hydrogels after culturing the cell-hydrogel constructs for 96 h.

FIG. 5. Modulating mineral morphology and spatial distribution of the mineralized pSBMA as a function of the chemical crosslinker content and the heating rate of the urea thermal decomposition-mediated mineralization process. Type I: isolated small spherical mineral nodules resulting from faster heating rates (1.0 and 0.5° C./min) or higher crosslinker contents (2.651 and 1.326 mol %); Type II: large spherical mineral nodules resulting from medium heating rates (0.2 and 0.1° C./min) or medium crosslinker contents (0.663, 0.265, and 0.133 mol %); Type III: stripe-like bicontinuous mineral aggregates of small spherical nodules resulting from slower heating rates (0.02° C./min) or lower crosslinker contents (<0.027 mol %).

FIG. 6. Effect of heating rate (overall mineralization time) on the pSBMA hydrogel mineralization outcomes. (a) μ-CT views (sagittal and coronal) of the mineralized hydrogels and the respective mineral size distribution as a function of the heating rate of the urea thermal decomposition. (b) SEM micrographs revealing the mineral nodule morphologies at the cross-sections of mineralized hydrogels as a function of the heating rate. (c) Mineral volume of the mineralized hydrogels (determined by μ-CT, n=3) as a function of the heating rate and the overall mineralization time. (d) Total calcium content of mineralized hydrogel (determined by selective ion electrode upon acid treatment, n=3) as a function of heating rate and overall mineralization time. (e) Mineral density of mineralized hydrogels (determined by μ-CT) as a function of heating rate. (f) Anisotropy of the mineralized hydrogels (determined by μ-CT, n=3) as a function of heating rate and overall mineralization time. (g) Mineral density distribution across the mineralized hydrogels as visualized by color mapping of sagittal cross-sections (by μ-CT) as a function of heating rate. Statistical significance was determined by one-way ANOVA with Tukey's multiple comparison. Pairwise comparisons of each parameter with those obtained at the fastest heating rate are statistically significant (P<0.05) unless denoted as NS (not significant). Complete pairwise comparisons are shown in Tables 4-7.

FIG. 7. Effect of chemical crosslinker content on the pSBMA hydrogel mineralization outcome. (a) μ-CT views (sagittal and coronal) of the mineralized hydrogels and respective mineral size distribution in diameters (determined by μ-CT) as a function of the crosslinker content. (b) SEM micrographs of the cross-sections of the mineralized hydrogels as a function of the crosslinker content. (c) Swelling ratios of pSBMA hydrogels by volume (S_{w}) in water (red) and in PBS (green) as a function of the chemical crosslinker content (n=3). The swelling ratios of pSBMA hydrogels in water by weight (S_{w}, black) and by volume (Sᵥ, red) are plotted in the inset. (d) Mineral volume of the mineralized hydrogels (determined by μ-CT, n=3) as a function of crosslinker content. (e) Total calcium content of the mineralized hydrogels (determined by selective ion electrode upon acid treatment, n=3) as a function of the crosslinker content. All hydrogels were mineralized with a heating rate of 0.2° C./min. Statistical significance was determined by one-way ANOVA with Tukey's multiple comparison. Pairwise comparisons of each parameter with those obtained with lowest crosslinker content (0.027 mol %) are statistically significant (P<0.05) unless denoted as NS (not significant). Complete pairwise comparisons are shown in Tables 9 & 10.

FIG. 8. Effects of prolonged mineralization (slower heating rate) vs. crosslinker content, as well as repeating mineralization cycles, on the mineralization outcome of pSBMA hydrogel. (a) SEM micrographs and μ-CT views of the sagittal cross-sections of the mineralized hydrogels with lower crosslinker content and shorter mineralization time (faster heating rate, left), lower crosslinker content and longer mineralization time (slower heating rate, middle), and higher crosslinker content and longer mineralization time (slower heating rate, right). (b) Mineral volume (determined by μ-CT, n=3) as a function of mineralization time (heating rate) at a low hydrogel crosslinker content of 0.027 mol %. (c) Mineral volume (determined by μ-CT, n=3) as a function of hydrogel crosslinker content with slower heating rate of 0.05° C./min (mineralization time of 19.3 h).

FIG. 9. Effect of repeated mineralization cycles on the mineralization outcome of pSBMA. (a) Sagittal μ-CT cross-section views, (b) mineral content, and (c) mineral nodule numbers (both by μ-CT, n=3) as a function of the number of repeated mineralization cycles. Statistical significance are determined by one-way ANOVA with Tukey's multiple comparison. All pairwise comparisons are significant (P<0.05) unless denoted as NS (not significant). The mineral content of the hydrogel that was subjected to 4 repeated mineralization cycles is equivalent to ˜60 wt % (mineral weight/dry composite weight).

FIG. 10. SEM micrographs revealing surface and cross-section mineral domain morphologies of the mineralized hydrogels containing identical crosslinker content (1.326 mol %) but varied side chain charges. All hydrogels were subjected to heterogeneous mineralization by controlled heating in an acidic solution of HA containing 2-M urea from 37° C. to 95° C. at a heating rate of 0.2° C./min.

FIG. 11. HR-TEM micrograph of the mineralized composite formed at the heating rate of 0.2° C./min, suggesting predominantly crystalline phase of the mineral component.

FIG. 12. Cell viability of rMSC encapsulated by pSBMA and the PEGMA control (with identical crosslinker content of 1.33 mol %) as determined by MTT assay (n=3). No significant difference (p>0.05) was observed between the cells encapsulated by the 2 hydrogel formulations at a given time, while significant increases in viable cells were detected over time in each hydrogel formulation Statistical significance was determined by one-way ANOVA with Tukey's multiple comparison.

FIG. 13. SEM micrographs of the surfaces and cross-sections of mineralized pSBMA hydrogels as a function of the heating rate. All mineralized hydrogel specimens contained the same crosslinker content of 0.265 mol %.

FIG. 14. Reconstructed μ-CT images of mineralized pSBMA hydrogels obtained with various heating rates. All mineralized hydrogel specimens contained the same crosslinker content of 0.265 mol %.

FIG. 15. Temporal pH changes of the mineralization solution as a function of the heating rate and overall mineralization time (n=3). The first pH plateaus (from pH 3.8 to 4.0, indicated by the green box) observed in all six pH-mineralization time curves could be attributed to the deprotonation of H₂PO₄⁻ ions. All specimens contained the same hydrogel crosslinker content of 0.265 mol %.

FIG. 16. SEM micrographs revealing the different mineral nodule sizes and morphologies as a function of the heating rate. a & e, Specimen mineralized at 0.5° C./min where small spherical mineral nodules were formed (Type I). b & f, Specimen mineralized at 0.2° C./min where large spherical mineral nodules were formed (Type II). c & g, Specimen mineralized at 0.05° C./min where larger spherical mineral domains containing multiple smaller nodules were formed, indicating a departure from a typical Type II mineral morphology. d & h, Specimen mineralized at 0.02 ° C/min where bicontinuous stripe-like mineral aggregates were formed (Type III). All mineralized specimens contained the same crosslinker content of 0.265 mol %.

FIG. 17. Digital photograph images of the mineralized pSBMA hydrogels at various time points of the acid treatment. All mineralized hydrogel specimens contained the same crosslinker content of 0.265 mol %.

FIG. 18. Compressive mechanical properties of the mineralized pSBMA hydrogels as a function of the heating rate. a) Compressive stress-strain curves of hydrated specimens as a function of the heating rate of their mineralizations. The data were recorded on a Q800 DMA equipped with a submersion compression fixture at 25° C. b) Stress at 50% compressive strain of the mineralized specimens as a function of the heating rate and the respective mineralization time. Statistical significance was determined by one-way ANOVA with Tukey's multiple comparison tests. Pairwise comparisons with unmineralized pSBMA hydrogel are statistically significant (P<0.05) unless denoted as NS (not significant). Complete pairwise comparisons are in shown in Table 8. All mineralized hydrogel specimens contained the same crosslinker content of 0.265 mol %.

FIG. 19. Digital photograph images of the mineralized hydrogels with different crosslinker contents at various time points of the acid treatment. All the hydrogel specimens were mineralized with a heating rate of 0.2° C./min.

FIG. 20. Reconstructed μ-CT images of mineralized pSBMA hydrogels containing different crosslinker content. All specimens were mineralized with a heating rate of 0.2° C./min.

FIG. 21. SEM micrographs of the surfaces and cross-sections of the mineralized pSBMA hydrogels containing different crosslinker contents. All hydrogel specimens were mineralized with a heating rate of 0.2° C./min.

FIG. 22. Mineral density (a) distribution colored mapping (b) of pSBMA hydrogels as a function of crosslinking content (determined by μ-CT). The mineral density increased gradually with the increase of crosslinker content (obviously from 0.027 to 0.265 mol %, but not so clear from 0.265 to 2.651 mol % due to the mineral size). All specimens were mineralized with a heating rate of 0.2° C./min. Complete pairwise comparisons of mineral density are shown in Table 11.

FIG. 23. Bone growth over a 5-mm rat femoral segmental defect guided by PSBMA hydrogel. (a) A pSBMA hydrogel (5 mm×3 mm×3 mm) press-fit within the femoral segmental defect stabilized by a radiolucent polyetheretherketone (PEEK) plate fixator. (b) Reconstructed μ-CT 3D images & 2D bone mineral density color mapping of the center longitudinal slice of the defect treated with PSBMA hydrogel grafts at 4 and 12 weeks post-op. (c) Bone volume & (d) Bone mineral density of the defects (n=4) treated with pSBMA hydrogel grafts at 4, 8 and 12 weeks post-op. *p<0.05 (two-way ANOVA).

### DESCRIPTION OF THE INVENTION

This invention provides novel compositions and methods for truly three-dimensional mineralization templated by synthetic scaffolds and facilitated by zwitterionic mediators. The invention enables 3-D mineralization nucleation and growth of minerals in a well-controlled and defined manner. For example, the zwitterionic hydrogels of the invention are suited for thermal and pH-driven mineralization process where the experimental parameters such as heating rate, rate of pH changes, and overall mineralization time can be designed to control the mineral density, spatial distribution, orientation and morphology of the template mineral growth. The composite materials prepared by the disclosed methods are cytocompatible and/or biodegradable and are suitable for use as medical implants in a wide range of orthopedic and dental applications. The invention enables effective in vitro mineralization (e.g. for improving scaffolds' osteoconductivity) and in vivo osteointegration for scaffold-assisted skeletal tissue repair.

It is demonstrated herein that zwitterionic sulfobetaine is effective in templating extensive heterogeneous HA-mineralization across 3-D hydrogel scaffolds. The uniquely positioned opposite charges have likely played a critical role in facilitating active infiltration of oppositely charged precursor ions or pre-nucleation ion clusters and lowering the interfacial energy for heterogeneous nucleation and growth. (Nudelman, et al. 2010 Nat Mater 9:1004-9; Koumoto, et al. 2004 Biomaterials 25:3915-21.)

The significantly more cytocompatible pSBMA, along with its appropriate (not excessive) swelling behavior under physiological conditions, make the zwitterionic hydrogel uniquely suited for in vivo applications and for the preparation of higher density composites. The end-stage the mineralization outcome of the zwitterionoic hydrogel, including size, density, and the spatial distribution of the templated mineral growth, can be readily controlled through facile adjustments of the heating rate of the urea thermal decomposition-mediated mineralization process and the crosslinker content of the zwitterionic hydrogels. The inexpensive sulfobetaine can be incorporated into a wide variety of synthetic scaffolds, offering greater versatility than natural organic extracellular matrices (ECM) such as collagen for scaffold-assisted skeletal tissue repair.

The zwitterionic ligands can be incorporated into a wide variety of synthetic scaffolds including self-assembling polymers and block copolymers, either covalently or non-covalently crosslinked and/or associated.

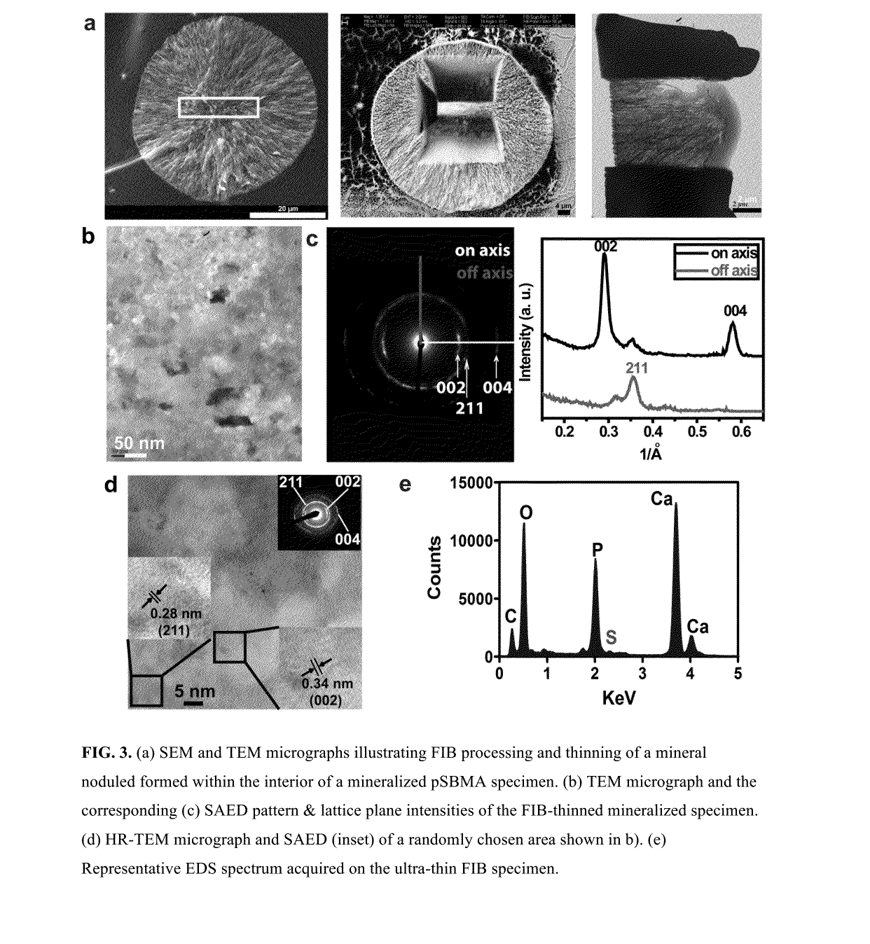

An important feature of the invention is that it enables structurally integrated mineralization. For example, the mineral nodules formed within the interior of pSBMA hydrogel were structurally well-integrated with the templating hydrogel matrix, as evidenced by SEM micrographs of the cross-sections of the composite (FIG. 3a), where no delamination of the spherical mineral nodules from the hydrogel matrix was observed when the air-dried composites were fractured to expose the cross-sections. Further, the EDS analysis of the ultra-thin FIB specimens of the mineral nodules detected the S signal distinctively associated with the sulfobetaine side chains of the pSBMA (FIG. 3e), along with the Ca and P signals associated with the HA crystals, further confirming the structural integration of the mineral nodule and its templating zwittertionic polymer chain.

In one aspect, the invention generally relates to a structurally well-integrated composite material comprising 3-dimensioanl network of a synthetic polymer that includes a zwitterionic moiety and structurally integrated mineral components throughout the 3-dimensional network.

In certain embodiments, the 3-D polymer network includes a copolymer, e.g., a block cocolymer.

In certain preferred embodiments, the 3-D polymer network is crosslinked, forming a 3-D crosslinked polymer network, such as a crosslinked hydrogel of polymethacrylate or polyacrylate. For example, the 3-D polymer network may have a crosslinking density from about 0.01 mol % to about 10 mol % (e.g., about 0.01 mol % to about 5 mol %, about 0.01 mol % to about 3 mol %, about 0.01 mol % to about 2 mol %, about 0.01 mol % to about 1 mol %, about 0.02 mol % to about 5 mol %, about 0.02 mol % to about 3 mol %, about 0.02 mol % to about 2 mol %, about 0.5 mol % to about 10 mol %, about 0.5 mol % to about 5 mol %, about 0.5 mol % to about 3 mol %, about 0.1 mol % to about 5 mol %, about 0.1 mol % to about 3 mol %, about 0.1 mol % to about 2 mol %).

In certain preferred embodiments, the repeating structural unit has a pendent zwitterionic moiety. The zwitterionic moieties used may be any suitable zwitterionic moieties such as sulfobetaine, phosphorylcholine, and carboxybetaine. In certain preferred embodiments, the zwitterionic moiety is sulfobetaine.

The sulfobetaine has the structure:

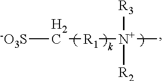

wherein R₁ is —CH₂—, —O—CH₂—, or —O—C₂H₅—, k is an integer from about 0 to about 15, each R₂ and R₃ is independently an alkyl group (e.g., methyl, ethyl, other C₁-C₁₂ alkyl groups).

Phosphobetaine has the structure of

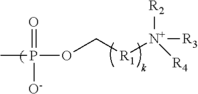

wherein R₁ is —CH₂—, —O—CH₂—, or —O—C₂H₅—, k is an integer from about 0 to about 15, each R₂, R₃ and R₄ is independently an alkyl group (e.g., methyl, ethyl, other C₁-C₁₂ alkyl groups).

Carboxybetaine has the structure:

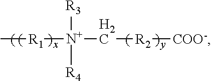

wherein each R₁ and R₂ is independently —CH₂—, —O—CH₂—, or —O—C₂H₅—, x is an integer from about 1 to about 15, y is an integer from about 0 to about 15, each R₃ and R₄ is independently an alkyl group (e.g., methyl, ethyl, other C₁-C₁₂ alkyl groups).

The mineral components include any suitable material (synthetic or natural) such as bone mineral, e.g., hydroxyapatite, substituted hydroxyapatites (e.g., carbonated, halogenated, metal ion-substituted), calcium deficient hydroxyapatite, calcium apatite, calcium phosphates, octacalcium phosphate, tricalcium phosphate, and any transitional mineral phases between amorphous calcium phosphate to crystalline calcium apatite, and both amorphous and crystalline forms of calcium carbonate.

The composite material may have any suitable level of mineralization. In certain preferred embodiments, the composite material is characterized by a degree of mineralization from about 0.1 to about 95 wt % (e.g., from about 1 wt % to about 95 wt %, from about 1 wt % to about 70 wt %, from about 1 wt % to about 60 wt %, from about 1 wt % to about 40 wt %, from about 1 wt % to about 20 wt %, from about 5 wt % to about 95 wt %, from about 5 wt % to about 60 wt %, from about 10 wt % to about 95 wt %, from about 10 wt % to about 60 wt %, from about 20 wt % to about 95 wt %, from about 1 wt % to about 30 wt %, from about 1 wt % to about 20 wt %, from about 1 wt % to about 10 wt %, from about 5 wt % to about 70 wt %, from about 1 wt % to about 50 wt %, from about 5 wt % to about 40 wt %, from about 5 wt % to about 30 wt %, from about 5 wt % to about 20 wt %).

The composite material may have any suitable mineral domain morphology. In certain preferred embodiments, the composite material has a mineral domain morphology characterized with isolated mineral nodules (e.g., spherical or substantially spherical) having a dimension from about 1 μm to about 300 μm (e.g., from about 1 μm to about 250 μm, from about 1 μm to about 200 μm, from about 1 μm to about 150 μm, from about 1 μm to about 100 μm, from about 1 μm to about 50 μm, from about 5 μm to about 300 μm, from about 10 μm to about 300 μm, from about 50 μm to about 300 μm, from about 100 μm to about 300 μm, from about 5 μm to about 200 μm, about 5 μm, 10 μm, 25 μm, 50 μm, 100 μm, 150 μm, 200 μm, 250 μm)(including Type I and Type II mineral domain morphologies).

The composite material may have any suitable mineral domain morphology. In certain preferred embodiments, the composite material has a Type IIII mineral domain morphology characterized with substantially continuous mineral aggregates of constituting mineral nodules (e.g., spherical or substantially spherical), wherein the constituting mineral nodules have a dimension from about 1 μm to about 300 μm (e.g., about 5 μm, 10 μm, 25 μm, 50 μm, 100 μm, 150 μm, 200 μm, 250 μm).

The composite material may further include encapsulated cells (e.g., live cells). For example, the cells comprise stem or progenitor cells. In certain preferred embodiments, the cells are selected from bone marrow stromal cells. In certain embodiments, the 3-D mineralized synthetic scaffold is capable of supporting attachment and/or encapsulation of cells. In certain preferred embodiments, the 3-D mineralized synthetic scaffold is capable of supporting attachment and/or encapsulation of a biological agent.

In certain preferred embodiments, the composite material is cytocompatible. In certain preferred embodiments, the composite material is biodegradable.

In yet another aspect, the invention generally relates to a method for mediating 3-dimensional mineralization. The method includes (1) providing a mixture that includes: (a) a 3-dimensional scaffold of a polymer network, wherein the polymer network comprises a zwitterionic moiety; (b) a mineral precursor; and (c) a pH mediator; (2) causing the pH mediator to gradually adjust the pH level of the mixture; and (3) transforming the mineral precursor into mineral deposits as templated the 3-dimensinal scaffold.

In certain embodiments, causing the pH mediator to gradually adjust the pH level of the mixture is by decomposing the mineral precursor to generate OH⁻ or H⁺. The pH mediator may be any suitable compound or agent, including for example, urea, ammonia, or ammonium hydroxide. In certain preferred embodiments, the pH mediator is urea and causing the pH mediator to gradually adjust the pH level of the mixture comprises thermally decomposing urea to generate OH⁻. In certain preferred embodiments, thermally decomposing urea comprises raising temperature of the mixture at a rate of about 0.001° C./min to about 1° C./min (e.g., about 0.005° C./min, 0.01° C./min, 0.02° C./min, 0.05° C./min, 0.1° C./min, 0.2° C./min, 0.3° C./min, 0.4° C./min, 0.5° C./min, 0.6° C./min, 0.7° C./min, 0.8° C./min, 0.9° C./min).

The mineral precursor is selected from calcium apatites, hydroxyapatite, substituted hydroxyapatites (e.g. carbonated, halogenated, metal ion substituted), calcium deficient hydroxyapatite, calcium phosphates, octacalcium phosphate, tricalcium phosphate, and any transitional mineral phases between amorphous calcium phosphate to crystalline calcium apatite, and both amorphous and crystalline forms of calcium carbonate.

The invention also relates to a 3-D mineralized synthetic scaffold prepared according to a method disclosed herein.

In certain preferred embodiments, the 3-D mineralized synthetic scaffold is capable of supporting attachment and/or encapsulation of cells. In certain preferred embodiments, the 3-dimensional mineralized synthetic scaffold is capable of supporting attachment and/or encapsulation of a biological agent.

In certain preferred embodiments, the 3-dimensional mineralized synthetic scaffold is suitable for implant as a replacement material for dental, bone, cartilage, tendon, ligament, or osteochondral damage.

In yet another aspect, the invention generally relates to an implant having a composite material that is characterized by a 3-D scaffold of a polymer network substantially uniformly mineralized therein hydroxyapatite crystals. The polymer includes a structural repeating unit having a zwitterionic moiety.

In yet another aspect, the invention generally relates to a 3-dimensional scaffold that includes a 3-dimensional polymer network. The polymer network includes a zwitterionic moiety. In certain preferred embodiments, the 3-dimensional scaffold is suitable as mineralization templates either in vitro or in vivo.

In certain preferred embodiments, the composite material is cytocompatible. In certain preferred embodiments, the composite material is biodegradable.

The implant may be polymeric or metallic implants, screws, fixators and surgical devices or a component thereof, such as a catheter, a vascular stent, a dental implant, an orthopedic implant, an implant of bone, cartilage, tendon, ligament, osteochondral replacement. Exemplary bone, joint, and dental metallic implants include: fixation plates, IM rods, screws, total joint replacement prosthetics; dental filler/composites; spine fusion metallic cages. Exemplary orthopedic implants include: an implant for total knee replacement (TKR), total hip replacement (THR), total shoulder replacement (TSR), total elbow replacement (TER), total wrist replacement (TWR), total ankle replacement (TAR), or a component thereof

For certain applications, the implant may further include attached and/or encapsulated live cells (e.g., stem or progenitor cells).

The crosslinking density of zwitterionic hydrogels can be used to control mineral density, spatial distribution, orientation and morphology of the template mineral growth.

### Bone Mineralization

Carbonated calcium-deficient hydroxyapatite is the main mineral of which dental enamel and dentin are comprised. Up to 70% of bone by weight is a modified form of hydroxyapatite, which can be found in teeth and bones within the human body. HA is commonly used as a filler to replace amputated bone or as a coating to promote bone ingrowth into prosthetic implants. HA (Ca₁₀(PO₄)₆(OH)₂) is a member of the family of calcium apatites Ca₁₀(PO₄)₆X₂, with X═OH, Cl, F or Br, and it can be found in the nature on hexagonal and monoclinic phases. HA is the principal constituent of the bone mineral phase. Hydrolysis of urea may be used to facilitate the generation of hydroxyl ion (OH⁻), which induces the transformation of HPO₄²⁻ into PO₄³⁻. With the pH increasing the HA crystals precipitate.

(NH₂)₂CO+H₂O→2NH₃+CO₂

2NH₃+H₂O→2NH₄⁺+OH⁻

According to the classical descriptions of activation energy of homogenous nucleation, nucleation rate, and mineral growth rate for a spherical nucleus, increasing supersaturation (S) and decreasing interfacial free energy (a) expedite the rate of the nucleation and growth (Equations 1-3). (Mann S. 2001 Biomineralization: Principles and Concepts in Bioinorganic Materials Chemistry. Oxford: Oxford University Press.) For a spherical nucleus, the activation energy of homogeneous nucleation ΔG_{N}*, nucleation rate J_{N}, and mineral growth rate J_{G} can be described as Equations 1, 2 and 3 (Mann S. Biomineralization: Principles and Concepts in Bioinorganic Materials Chemistry. Oxford: Oxford University Press; 2001.):

ΔG_{N}*=16πσ³v²/3(kT InS_{R})² Eq-1

where σ is interfacial free energy per unit surface area, v is molecular volume, S_{R} is relative supersaturation, k is the Boltzmann constant, T is absolute temperature.

J_{N}=A exp[−ΔG_{N}*/kT] Eq-2

where A is a pre-exponential factor.

J_{G}=k₂(S_{A})^{x} Eq-3

where, k₂ is a rate constant, S_{A} is the absolute supersaturation, and x≧1

These parameters similarly govern heterogeneous nucleation and growth. In nature, heterogeneous mineralization of ECM in calcified tissues are accomplished by the tight control over ion transport to achieve appropriate local supersaturation (e.g., within diffusion-limited cellular and/or extracellular spaces) and by complex organic ECM templates that stabilize precursor ions, reduce the interfacial energy, and modulate the heterogeneous nucleation and mineral growth. (Mann 1988 Nature 332:119-24; Weiner, et al. 1998 Annu Rev Mater Sci 28:271-98; Traub, et al. 1989 Proc Natl Acad Sci USA 86:9822-6; George, et al. 2008 Chem Rev 108:4670-93.)

For instance, both collagen fibrils in bone and the acidic non-collagenous proteins (NCPs) attached to the collagen matrix are believed to play important roles in facilitating precursor ion infiltration and creating local supersaturation, spatially confining and stabilizing initial amorphous calcium phosphate deposition (ACP), and templating/modulating subsequent transformation into more stable crystalline phases. (Weiner, et al. 1998 Annu Rev Mater Sci 28:271-98; Traub, et al. 1989 Proc Natl Acad Sci USA 86:9822-6. Hunter, et al. 1994 Biochemical Journal 302:175-9; Tye, et al. 2003 J Biol Chem 278:7949-55; He, et al. 2003 Nature Materials 2:552-8; Veis, et al. 1967 Biochemistry 6:2409-16; George, et al. 1996 J Biol Chem 271:32869-73; Olszta, et al. 2007 Mat Sci Eng R 58:77-116; Jahnen-Dechent, et al. 2011 Circ Res 108:1494-509; Boskey, et al. 2002 Calcified Tissue Int 71:145-54; Fantner, et al. 2005 Nat Mater 4:612-6; Gebauer, et al. 2011 Nano Today 6:564-84; Mahamid, et al. 2010 Proc Natl Acad Sci USA 107:6316-21.)

A recent study revisiting the role of collagen in templating mineralization in the presence of HA nucleation inhibitors suggests that the positive net charge close to the C-terminal end of the collagen molecules promotes the infiltration of ACP, and that the clusters of charged amino acids, both in gap and overlap regions of collagen fibrils, form nucleation sites mediating the transformation of ACP into oriented apatite crystals. (Nudelman, et al. 2010 Nat Mater 9:1004-9.)

Zwitterionic materials, well known for excellent anti-fouling ability, have been extensively explored for applications ranging from blood compatible materials to DNA and protein delivery vehicles. (Banerjee, et al. 2011 Advanced Materials 23:690-718; Chen, et al. 2010 Polymer 51:5283-93; Ishihara, et al. 1998 J Biomed Mater Res 39:323-30; Yan, et al. 2007 Sci China Ser B 50:660-4; Nakaya, et al. 1999 Prog Polym Sci 24:143-81; Liu, et al. 2010 J Membrane Sci 350:387-94; Jiang, et al. 2010 Adv Mater 22:920-52; Andrew, et al. 2012 Nature Chemistry 4:59-63.) Although calcium phosphate nanocomposite of polyelectrolytes and zwitterionic polymers were previously prepared by direct mixing of the mineral components or solidification of precursor ion solutions with the respective polymers, the role of zwitterionic ligands in templating heterogeneous nucleation and growth of HA has not been previously explored. (Stupp, et al. 1992 J Biomed Mater Res 26:169-83; Stupp, et al. 1993 J Biomed Mater Res 27:289-99; Kanapathipillai, et al. 2008 Chem Mat 20:5922-32.) Zwitterionic polymers, possessing equal number of oppositely charged residues in the same molecular side chain yet overall electrically neutral, are known to swell more in salt solution than in water, a behaviour opposite to those exhibited by anionic or cationic polyelectrolytes and driven by salt ion-mediated disruption of the extensive physical (electrostatic) crosslinking of the zwitterionic side chains formed in water (FIG. la; note the substantial difference in dipole moment: ˜24 Debye for sulfobetaine vs. 2 Debye for water). (Galin, et al. 1993 J Chem Soc-Perkin Trans 2 545-53; Clough, et al. 1973 J Chem Phys 59:2254-9.)

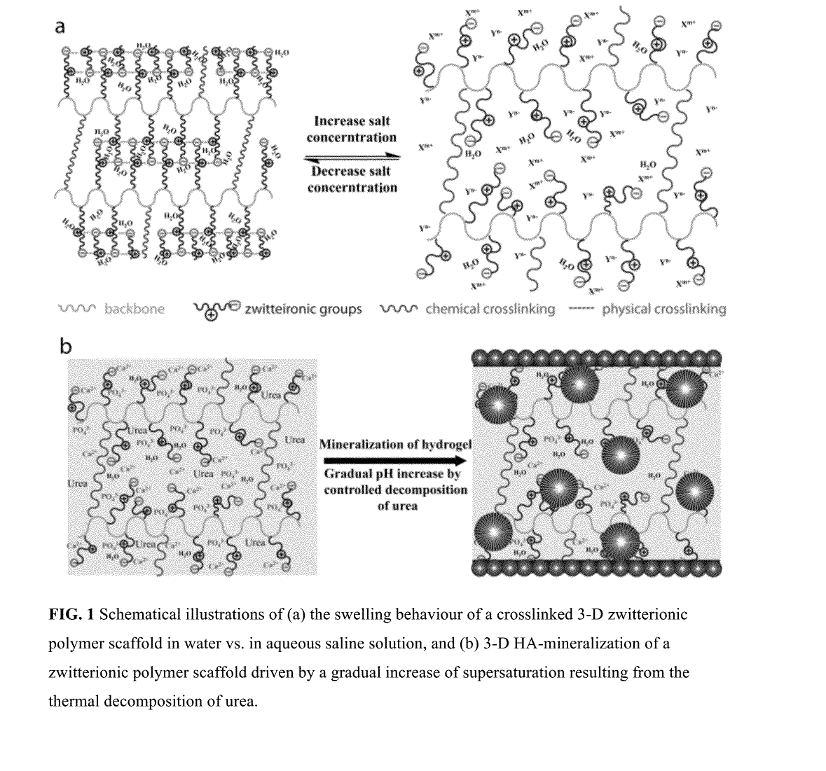

The invention takes advantage of such antipolyeletrolyte behavior exhibited by 3-D zwitterionic scaffolds, coupled with the ability of zwitterioic ligands to recruit and retain both cationic and anionic precursor ions or ion clusters and lower the activation energy for heterogeneous nucleation and growth, can enable more extensive 3-D HA-mineralization (FIG. 1b). (Lee, et al. 1998 J Appl Polym Sci 69:2021-34.)

The potency of photo-crosslinked polymethacrylate hydrogels were examined and compared that have zwitterionic sulfobetaine, cationic tertiary amine, anionic sulfonic acid or uncharged hydroxyl side chain residues in templating 3-D HA-mineralization. The relative cytocompatibility of zwitterionic, cationic and anionic scaffolds were also investigated by direct encapsulation of bone marrow stromal cells within these hydrogel scaffolds. Further, practical controls over the mineralization outcome, including size, content, and density of the mineral growth within the 3-D zwitterionic scaffold, were systematically investigated by altering crosslinker contents of the hydrogel and the external experimental parameters governing the heterogeneous mineralization process.

### Zwitterionic Sulfobetaine as a Potent Mineral Nucleating Ligand

Zwitterionic ligands are shown herein to be effective mineral nucleating ligands and provide cytocompatible matrix for potential tissue engineering applications. Comparisons were made between the zwitterionic (pSBMA) with other charged or uncharged ligands. Similar polymethacrylate hydrogels bearing cationic (pTMAEMA), anionic (pSPMA), and polar uncharged (pHEMA) side chains that photo-crosslinked with identical crosslinker content (Table 1) were investigated. The photo-crosslinked polymethacrylate hydrogels were placed in the mineralization solution and subjected to heterogeneous HA-mineralization driven by gradual pH increases resulting from controlled thermal decomposition of urea upon heating from 37° C. to 95° C. at a rate of 0.2° C./min. (Song, et al. 2003 J Am Chem Soc 125:1236-43; Song, et al. 2005 J Am Chem Soc 127:3366-72.)

TABLE 1
Preparation of hydrogels with different side chain charges for
mineralization (No.1-4) and cyto-compatibility (No. 5-7) studies

| | | | | VA-086 | | Cell |
|---|---|---|---|---|---|---|
| | | Monomer | Crosslinker | stock | Milli-Q | culture |
| | | amount | amount | solution ^{c} | water | medium ^{d} |
| No. | Monomer | (mmol) | (μL) | (μL) | (μL) | (μL) |
| 1 | HEMA | 2 | 50 ^{a} | 100 | 1850 | 0 |
| 2 | SBMA | 2 | 50 ^{a} | 100 | 1850 | 0 |
| 3 | TMEAMA | 2 | 50 ^{a} | 100 | 1850 | 0 |
| 4 | SPMA | 2 | 50 ^{a} | 100 | 1850 | 0 |
| 5 | SBMA | 2 | 17.9 ^{b} | 100 | 0 | 1882.1 |
| 6 | TMEAMA | 2 | 35.8 ^{b} | 100 | 0 | 1864.2 |
| 7 | SPMA | 2 | 53.8 ^{b} | 100 | 0 | 1846.2 |

“^{a}” 10% (v/v) ethanol solution of EGDMA.
“^{b}” PEGDMA (Mₙ = 750; radical inhibitors were removed by passing through an aluminum oxide column). The amount of PEGDMA used for each hydrogel was adjusted to ensure comparable equilibrated volumes of pSBMA, pTMEAMA, and pSPMA gels in culture media upon cell encapsulation for the cytotoxicity study.
“^{c}” 2% (w/v) VA-086/PBS solution.
“^{d}” α-MEM supplemented with 20% FBS, 1% penicillin, 1% streptomycin, and 2% glutamine.

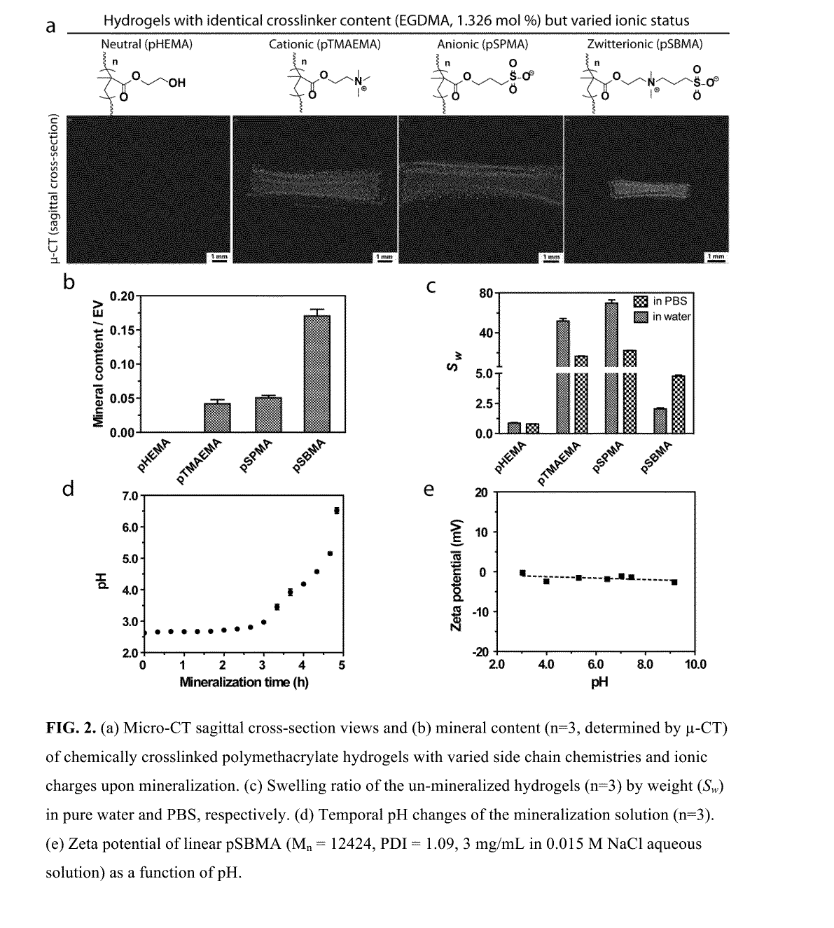

SEM and μ-CT analyses revealed that although substantial mineralization occurred on all hydrogel surfaces with varying surface mineral morphologies (FIG. 10), the degrees of mineralization within the interior of these 3-D hydrogel scaffolds varied significantly (FIG. 2a). Consistent with previous finding, the mineralization of the uncharged polar pHEMA hydrogel was limited to the surface of the hydrogel. (Song, et al. 2003 J Am Chem Soc 125:1236-43.) By contrast, the interior of cationic and anionic hydrogels were mineralized with spherical mineral nodules (FIG. 2b), likely benefiting from a combination of their exceptionally high swelling ratios that facilitated precursor ion infiltrations, and the ability of the charged residues to stabilize initial ACP and decrease the interfacial energy for subsequent nucleation and growth of mineral crystals. (Nudelman, et al. 2010 Nat Mater 9:1004-9.) A striking observation is that the zwitterionic hydrogel not only was substantially mineralized, but also exhibited much higher mineral content than its anionic and cationic counterparts mineralized under identical conditions (nearly 3 times higher in mineral content when normalized against their respective equilibrium volumes in PBS, FIG. 2c). This finding supports that zwitterionic hydrogels are more advantageous than both cationic/anionic and uncharged hydrogels in templating extensive 3-D mineralization. The sufficient (unlike pHEMA) yet not excessive (unlike pTMAEMA or pSPMA) swelling behavior of pSBMA in aqueous salt solutions as well as the ability of the zwitterionic ligands to recruit and stabilize oppositely charged precursor ions or ion clusters have likely made the difference in achieving 3-D mineralization with much higher mineral density. It is worth noting that within the range of pH increases during the urea thermal decomposition-mediated mineralization process (FIG. 2d), there is no significant fluctuation in zeta potential of the zwitterionic ligands as shown by the measurements taken with a linear pSBMA model polymer (FIG. 2e). This observation demonstrates that the ability of the zwitterionic sulfobetaine ligands within the crosslinked pSBMA hydrogel to facilitate active infiltration of oppositely charged precursor ions or ion clusters is maintained throughout the mineralization process. (Reichmuth, et al. 2003 J Chromatogr A 1013:93-101.)

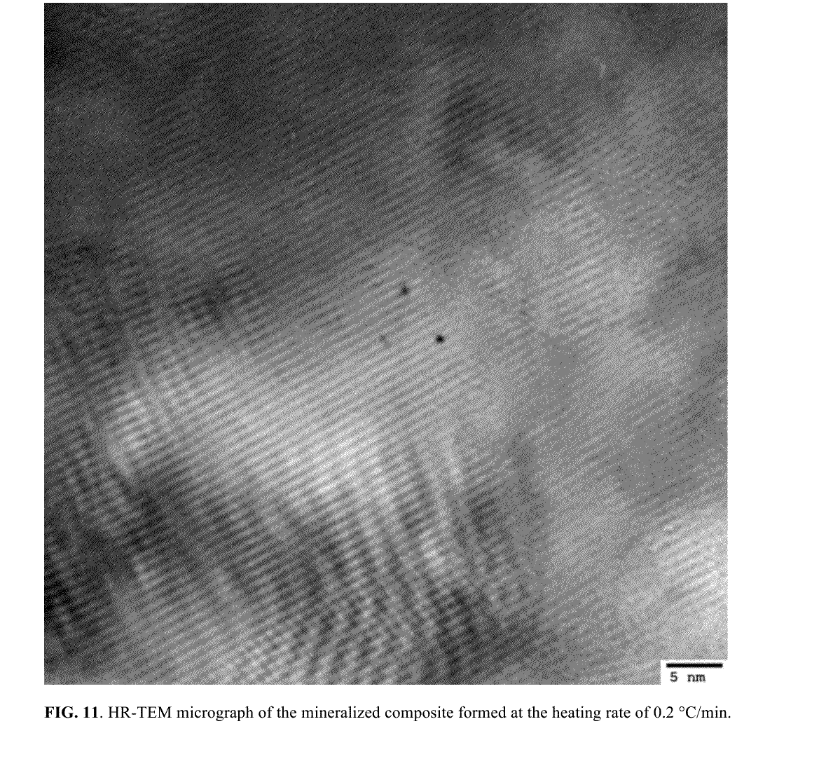

The mineral nodules formed within the interior of pSBMA were structurally well-integrated with the templating hydrogel matrix, as supported by SEM micrographs of the cross-sections of the composite (FIG. 3a). No delamination of the spherical mineral nodules from the hydrogel matrix was observed when the air-dried composites were fractured to expose the cross-sections. To examine the crystallinity and alignment of these mineral nodules at the nano-scale, the exposed cross-section was processed by focused ion beam to obtain an ultir-thin section of a typical spherical mineral nodule (FIB, FIG. 3a) and subjected to TEM, HR-TEM, SAED, and EDS analyses. The mineral nodule was predominantly crystalline in nature (FIG. 11), and the corresponding SAED of the TEM micrograph (FIG. 3b) exhibited more intense arcs corresponding to the (002) and (004) faces of HA than that corresponding to the (211) face (FIG. 3c), suggesting a preferential alignment of HA crystals along the c axis. The lattice spacings of 0.28 nm and 0.34 nm measured from the HR-TEM micrographs of randomly selected areas (FIG. 3d) correspond to the (002) and (211) faces of HA, agreeing with the intense diffraction ring patterns revealed by the SAED over the same areas (FIG. 3d inset). (Nelson, et al. 1984 Calcified Tissue Int 36:219-32; Suvorova, et al. 1999 J Microsc-Oxford 196:46-58.) Finally, S signal associated with the pSBMA hydrogel matrix (FIG. 3e) was consistently detected along with the Ca and P signals from the EDS spectra recorded on the ultra-thin FIB specimens (multiple randomly chosen areas were analysed for each specimen), further supporting structural integration of the mineral nodules with the templating organic matrix.

Cytocompatibility of Zwitterionic pSBMA Hydrogel vs. Cationic and Anionic Counterparts

The overall charge-neutral zwitterionic sulfobetaine ligands exhibit further advantage over polyelectrolyte mineral nucleating ligands in terms of cytocompatibility. To compare the cytotoxicity of these ligands imposed on skeletal progenitor cells, rat bone marrow stromal cells (rMSC) were encapsulated within pSBMA, pTMAEMA and pSPMA hydrogels crosslinked by poly(ethylene glycol) dimethacrylate (PEGDMA, Mₙ=750). The PEGDMA crosslinker was chosen for its aqueous solubility and well established cytocompatibility, which would help narrow down contributing factors to any observed cytotoxicity to the anionic, cationic or zwitterionic ligands. Given the dramatically different swelling behavior of these hydrogels, the PEGDMA crosslinker content was adjusted for each gel type (Table 1) to ensure comparable equilibrated cell-hydrogel construct volume in culture media (thus comparable 3-D cell encapsulation density). Live/dead cell straining performed after 96 h culture of the cell-hydrogel constructs revealed a significant number of dead cells in the cationic pTMAEMA hydrogel, consistent with previous reports on the cytotoxicity associated with polycationic scaffolds. (Zhu, et al. 2002 Biomaterials 23:4889-95; Hong, et al. 2006 Bioconjugate Chem 17:728-34.)

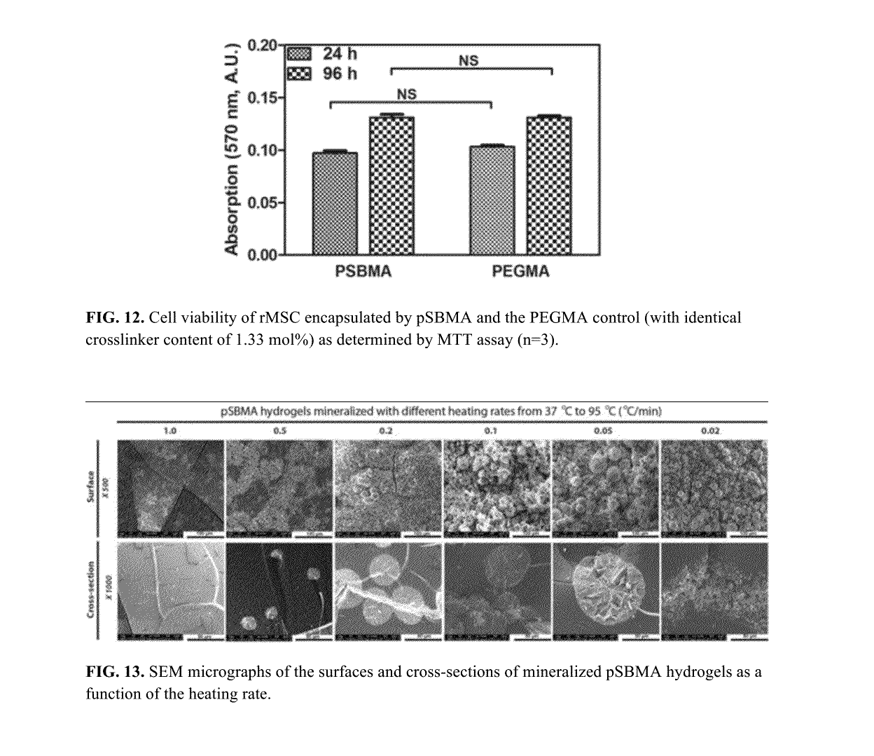

By contrast, MSCs encapsulated within the zwitterionic pSBMA hydrogel remained mostly viable after the 4-day culture (FIG. 4). Indeed, the viability and early proliferation of rMSCs encapsulated within the pSBMA hydrogel, determined by MTT cell viability assay, was comparable to those encapsulated within the cytocompatible PEGMA hydrogel control (FIG. 12). This observation is consistent with previous reports on the cytotoxicity associated with highly charged polyelectrolyte, particularly polycationic, scaffolds. (Zhu, et al. 2002 Biomaterials 23:4889-95; Hong, et al. 2006 Bioconjugate Chem 17:728-34.) These observations, combined with the ability of sulfobetaine ligands to template more extensive/higher-density, well-integrated 3-D mineralization of synthetic scaffolds than its charged or uncharged counterparts, indicating a promising role of this zwitterionic ligand in enabling improved osteointegration of scaffold-assisted skeletal tissue repair.

Regulating the HA-Mineralization Outcome of Zwitterionic pSBMA Hydrogels

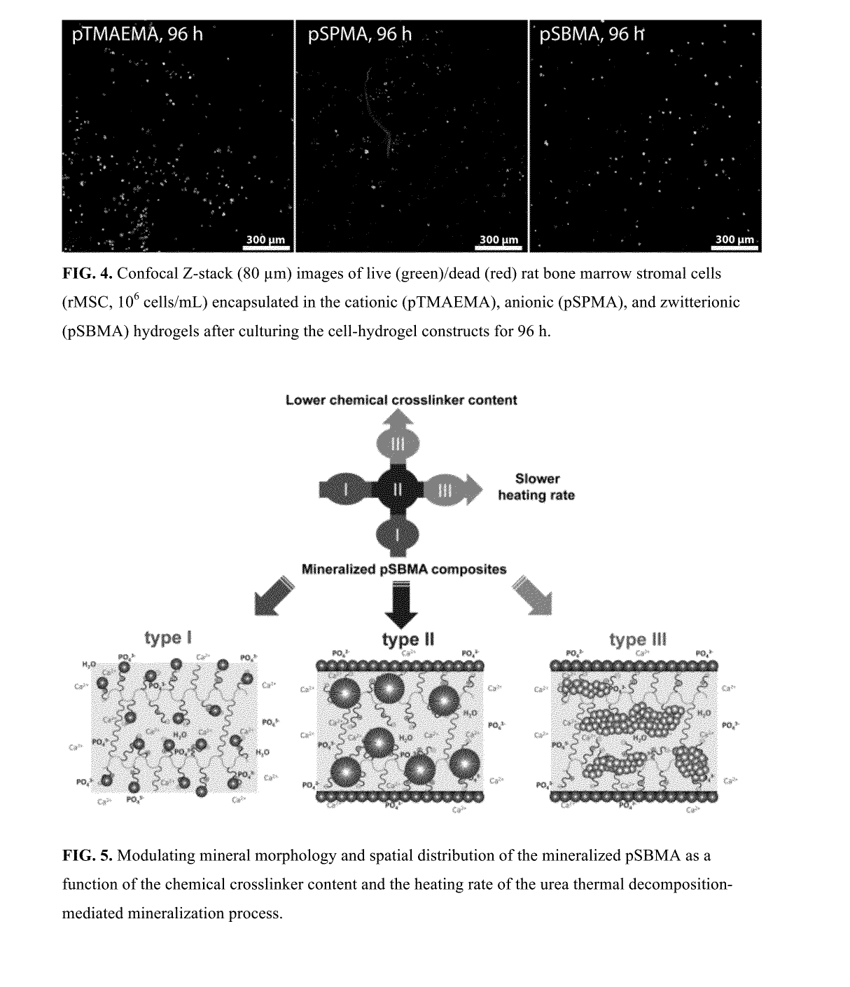

Further investigated was whether and how the size, spatial distribution and total mineral content of the mineral domains within the pSBMA hydrogel can be modulated. By altering the controlled heating rate of the urea decomposition-mediated heterogeneous mineralization process (thus the rate/degree of supersaturation) and the crosslinker content (thus the swelling ratio of the pSBMA hydrogel in high ionic-strength mineralization solution and the diffusibility of precursor ions), fine control over the in vitro mineralization outcome can be accomplished (Type I, II, and III, FIG. 5). Finally, by repeating an optimized mineralization process, the extent of the 3-D mineralization can be further improved.

### Regulating HA Mineralization Outcome by Heating Rate

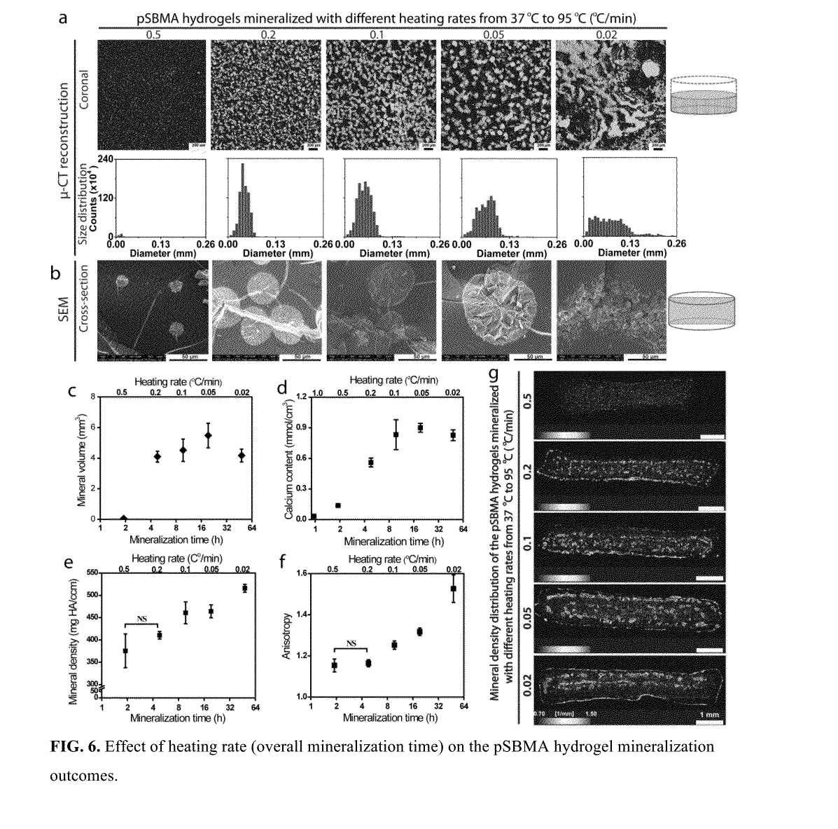

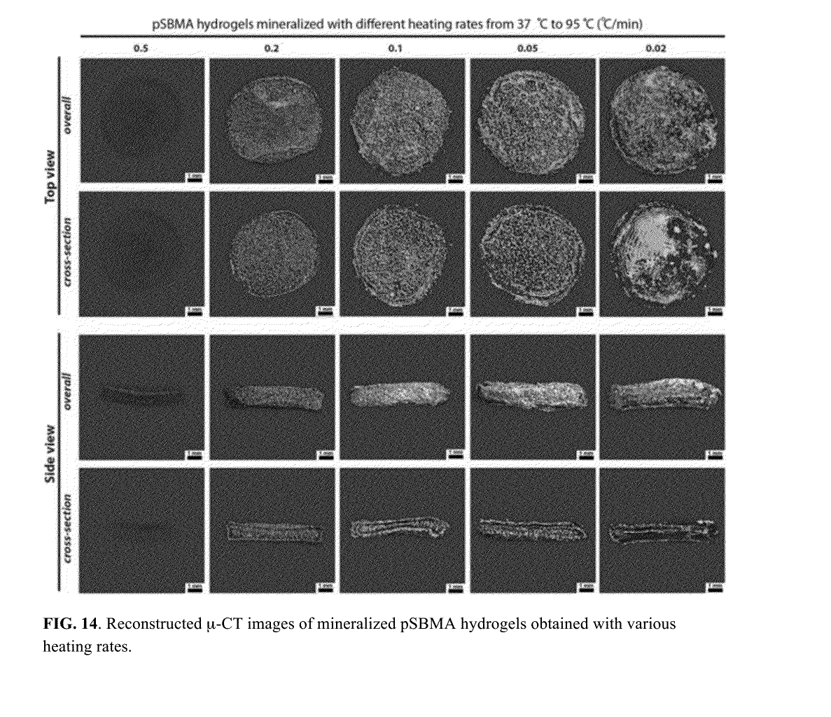

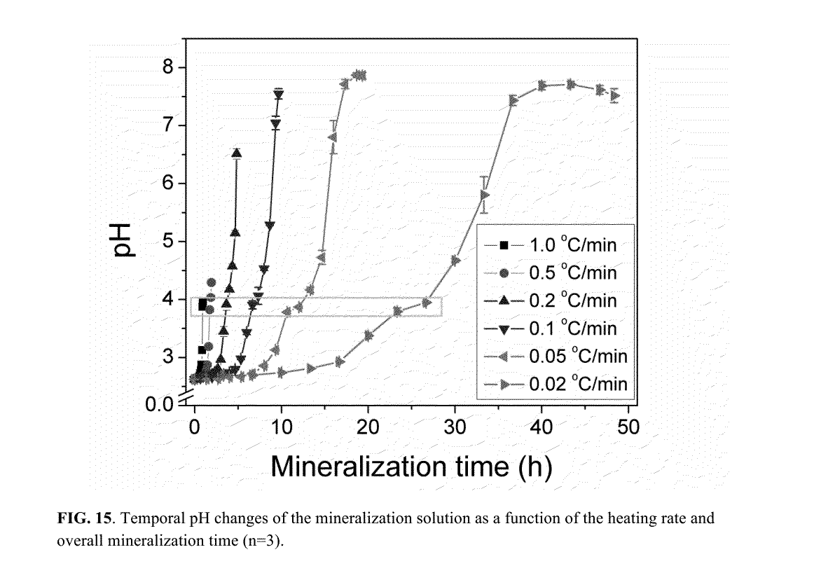

First demonstrated herein was that by altering the heating rate (0.02-1.0° C./min; Table 2), thus the rate of pH changes governing the supersaturation and the duration of the urea thermal decomposition-mediated mineralization process, the extent of mineralization within pSBMA hydrogel at a given crosslinker content (e.g. 0.265 mol % EGDMA) can be drastically altered (FIGS. 6a-g). (Nancollas, et al. 1994 Formation and dissolution mechanisms of calcium phosphates in aqueous systems. In: Brown P W, Constantz B, editors. Hydroxyapatite and Related Materials. Ann Arbor: CRC Press; p. 73-81.) At the fast heating rates of ≧0.5° C./min, the hydrogel was minimally mineralized (FIGS. 6a & 6b, FIGS. 13 & 14). This is likely due to the insufficient time (as a result of the fast pH increases revealed by pH-time plot in FIG. 15) for the rapidly supersaturated precursor ions or pre-nucleation ion clusters to diffuse into the hydrogel for heterogeneous nucleation and growth into stable micrometer-sized minerals identifiable by SEM and μ-CT. At heating rates of 0.1-0.5° C./min, however, the hydrogels could be substantially mineralized (FIGS. 6a & 6b, FIGS. 13 & 14), with the mineral volume and total calcium content (determined by μ-CT analysis in FIG. 6c and calcium release assays in FIG. 6d, respectively) increased with decreasing heating rates. More sufficient time for sustained mineral growth and more gradual pH increases resulting from the slower heating rates (FIG. 15) are primary contributing factors to the more robust mineralization throughout the 3-D scaffold. However, such an effect plateaued with further decreases of the heating rate, which did not lead to substantial continued increase in mineral content (FIGS. 6c & d).

TABLE 2
Preparation and mineralization conditions
of zwitterionic pSBMA hydrogels

| | | | | | Heat- | | |
|---|---|---|---|---|---|---|---|
| | SBMA | EGDMA | Cross- | I-2959 | ing | Miner- | |
| | Mon- | stock | linker | stock | rate | alization | Milli-Q |
| | omer | solution^{a} | content | solution^{b} | (° C./ | time^{c} | water |
| No. | (g) | (μL) | (mol %) | (μL) | min) | (min) | (μL) |
| 1 | 3.36 | 600 | 2.651 | 1200 | 0.2 | 290 | 660 |
| 2 | 3.36 | 300 | 1.326 | 1200 | 0.2 | 290 | 960 |
| 3 | 3.36 | 150 | 0.663 | 1200 | 0.2 | 290 | 1110 |
| 4 | 3.36 | 60 | 0.265 | 1200 | 0.2 | 290 | 1200 |
| 5 | 3.36 | 30 | 0.133 | 1200 | 0.2 | 290 | 1230 |
| 6 | 3.36 | 6 | 0.027 | 1200 | 0.2 | 290 | 1254 |
| 7 | 3.36 | 4.5 | 0.020 | 1200 | 0.2 | 290 | 1255.5 |
| 8 | 3.36 | 3 | 0.013 | 1200 | 0.2 | 290 | 1257 |
| 9 | 3.36 | 60 | 0.265 | 1200 | 1 | 58 | 1200 |
| 10 | 3.36 | 60 | 0.265 | 1200 | 0.5 | 116 | 1200 |
| 11 | 3.36 | 60 | 0.265 | 1200 | 0.2 | 290 | 1200 |
| 12 | 3.36 | 60 | 0.265 | 1200 | 0.1 | 580 | 1200 |
| 13 | 3.36 | 60 | 0.265 | 1200 | 0.05 | 1160 | 1200 |
| 14 | 3.36 | 60 | 0.265 | 1200 | 0.02 | 2900 | 1200 |
| 15 | 3.36 | 6 | 0.027 | 1200 | 0.05 | 1160 | 1254 |

“^{a}”10% (v/v) ethanol solution of EGDMA.
“^{b}”0.25% (w/v) water-acetone (19:1) solution of I-2959.
“^{c}”Total mineralization time from 37-95° C.

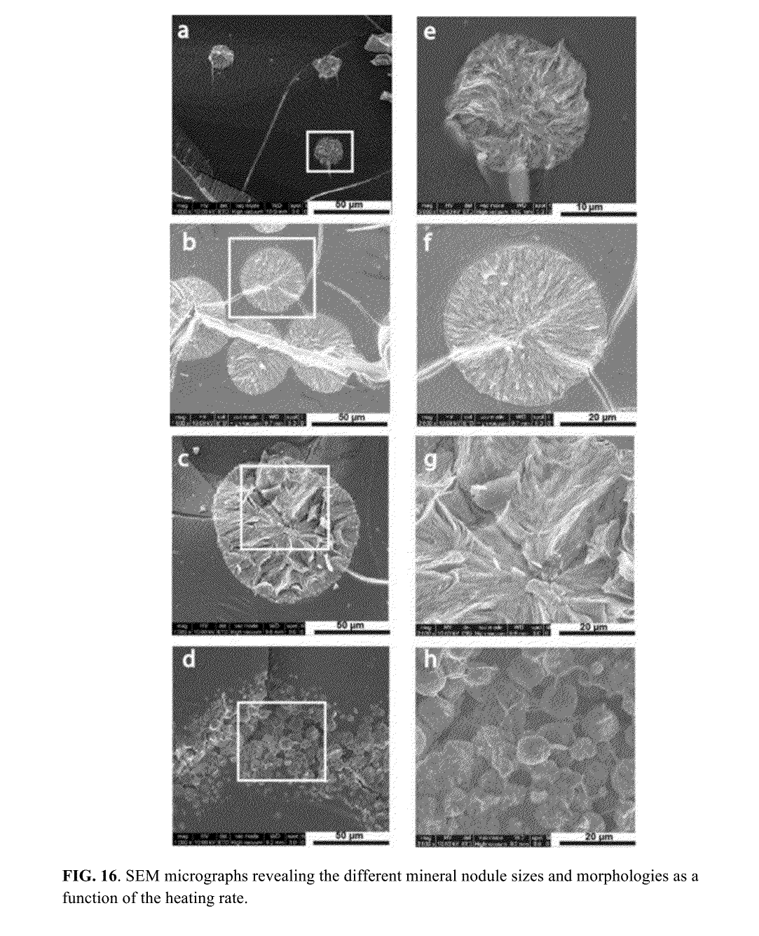

Interestingly, the size and spatial distribution of the mineral domains within the hydrogel also dramatically altered with the heating rate (FIGS. 6a & 6b, FIGS. 13 & 14). At the faster heating rates of 0.5-1.0° C./min, isolated small spherical mineral nodules (Type I, FIG. 5), characterized with a narrow mineral size distribution (8-24 μm; FIG. 6a), relatively low mineral density (FIGS. 6e & 6g) and low anisotropy (FIG. 6f), were formed across the hydrogel as supported by SEM micrographs (FIGS. 13, 16a & 16e). Although the higher supersaturation resulting from the faster heating (e.g. 0.5° C./min, FIG. 15) could expedite the heterogeneous nucleation and growth, the limited time (FIG. 15, Table 3) may have prevented adequate diffusion of precursor ions into the hydrogel to compensate the locally depleted precursor ion (due to mineral growth), resulting in the limited size of the mineral nodules. (Mann S. 2001 Biomineralization: Principles and Concepts in Bioinorganic Materials Chemistry. Oxford: Oxford University Press.)

TABLE 3
Sampling intervals for the monitoring of pH changes of
the mineralization solution with different heating rates

| | | | | | | |
|---|---|---|---|---|---|---|
| Heating rate (° C./min) | 1.0 | 0.5 | 0.2 | 0.1 | 0.05 | 0.02 |
| Overall mineralization | 58 | 116 | 290 | 580 | 1160 | 2900 |
| time (min) | | | | | | |
| Interval time (min) | 4 | 8 | 20 | 40 | 80 | 200 |
| Overall time points | 15 | 15 | 15 | 15 | 15 | 15 |

TABLE 4
Complete statistical significance data (p values) of the mineral volume
pairwise comparisons as a function of heating rate (corresponding to FIG. 5c)
as determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 0.2 vs 0.5 | 4.034 | 0.54536 | 10.46077 | 1.75E-04 | 0.05 | S | 2.23916 | 5.82884 |
| 0.1 vs 0.5 | 4.4537 | 0.54536 | 11.54912 | 7.47E-05 | 0.05 | S | 2.65886 | 6.24854 |
| 0.1 vs 0.2 | 0.4197 | 0.54536 | 1.08835 | 0.93377 | 0.05 | NS | −1.37514 | 2.21454 |
| 0.05 vs 0.5 | 5.41697 | 0.54536 | 14.04701 | 1.30E-05 | 0.05 | S | 3.62213 | 7.21181 |
| 0.05 vs 0.2 | 1.38297 | 0.54536 | 3.58624 | 0.15792 | 0.05 | NS | −0.41187 | 3.17781 |
| 0.05 vs 0.1 | 0.96327 | 0.54536 | 2.4979 | 0.44078 | 0.05 | NS | −0.83157 | 2.75811 |
| 0.02 vs 0.5 | 4.10743 | 0.54536 | 10.65119 | 1.50E-04 | 0.05 | S | 2.31259 | 5.90227 |
| 0.02 vs 0.2 | 0.07343 | 0.54536 | 0.19042 | 0.99991 | 0.05 | NS | −1.72141 | 1.86827 |
| 0.02 vs 0.1 | −0.34627 | 0.54536 | 0.89792 | 0.96568 | 0.05 | NS | −2.14111 | 1.44857 |
| 0.02 vs 0.05 | −1.30953 | 0.54536 | 3.39582 | 0.19198 | 0.05 | NS | −3.10437 | 0.48531 |

^{a}The different experimental groups are referred to as “X vs Y”, where X and Y stand for the respective heating rate (° C./min).
^{b}S indicates p < 0.05 (significant); NS indicates p > 0.05 (not significant).

TABLE 5
Complete statistical significance data (p values) of the calcium content pairwise
comparisons as a function of heating rate (corresponding to FIG. 5d) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 0.5 vs 1 | 0.1052 | 0.06855 | 2.17039 | 0.65095 | 0.05 | NS | −0.12505 | 0.33545 |
| 0.2 vs 1 | 0.52673 | 0.06855 | 10.8671 | 6.48E-05 | 0.05 | S | 0.29649 | 0.75698 |
| 0.2 vs 0.5 | 0.42153 | 0.06855 | 8.69671 | 5.45E-04 | 0.05 | S | 0.19129 | 0.65178 |
| 0.1 vs 1 | 0.7996 | 0.06855 | 16.49664 | 7.08E-07 | 0.05 | S | 0.56935 | 1.02985 |
| 0.1 vs 0.5 | 0.6944 | 0.06855 | 14.32625 | 3.60E-06 | 0.05 | S | 0.46415 | 0.92465 |
| 0.1 vs 0.2 | 0.27287 | 0.06855 | 5.62955 | 0.01752 | 0.05 | S | 0.04262 | 0.50311 |
| 0.05 vs 1 | 0.8674 | 0.06855 | 17.89543 | 3.24E-07 | 0.05 | S | 0.63715 | 1.09765 |
| 0.05 vs 0.5 | 0.7622 | 0.06855 | 15.72504 | 1.26E-06 | 0.05 | S | 0.53195 | 0.99245 |
| 0.05 vs 0.2 | 0.34067 | 0.06855 | 7.02834 | 0.0034 | 0.05 | S | 0.11042 | 0.57091 |
| 0.05 vs 0.1 | 0.0678 | 0.06855 | 1.39879 | 0.91288 | 0.05 | NS | −0.16245 | 0.29805 |
| 0.02 vs 1 | 0.7942 | 0.06855 | 16.38524 | 7.70E-07 | 0.05 | S | 0.56395 | 1.02445 |
| 0.02 vs 0.5 | 0.689 | 0.06855 | 14.21484 | 3.92E-06 | 0.05 | S | 0.45875 | 0.91925 |
| 0.02 vs 0.2 | 0.26747 | 0.06855 | 5.51814 | 0.02001 | 0.05 | S | 0.03722 | 0.49771 |
| 0.02 vs 0.1 | −0.0054 | 0.06855 | 0.11141 | 1 | 0.05 | NS | −0.23565 | 0.22485 |
| 0.02 vs 0.05 | −0.0732 | 0.06855 | 1.5102 | 0.88491 | 0.05 | NS | −0.30345 | 0.15705 |

^{a}The different experimental groups are referred to as “X vs Y”, where X and Y stand for the respective heating rate (° C./min).
^{b}S indicates p < 0.05 (significant); NS indicates p > 0.05 (not significant).

TABLE 6
Complete statistical significance data (p values) of the mineral density pairwise
comparisons as a function of heating rate (corresponding to FIG. 5e) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 0.2 vs 0.5 | 34.85333 | 21.91505 | 2.24914 | 0.53417 | 0.05 | NS | −37.2709 | 106.9775 |
| 0.1 vs 0.5 | 85.19043 | 21.91505 | 5.49748 | 0.01986 | 0.05 | S | 13.06623 | 157.3146 |
| 0.1 vs 0.2 | 50.3371 | 21.91505 | 3.24833 | 0.22254 | 0.05 | NS | −21.7871 | 122.4613 |
| 0.05 vs 0.5 | 88.58147 | 21.91505 | 5.71631 | 0.01567 | 0.05 | S | 16.45726 | 160.7057 |
| 0.05 vs 0.2 | 53.72813 | 21.91505 | 3.46716 | 0.17853 | 0.05 | NS | −18.3961 | 125.8523 |
| 0.05 vs 0.1 | 3.39103 | 21.91505 | 0.21883 | 0.99985 | 0.05 | NS | −68.7332 | 75.51524 |
| 0.02 vs 0.5 | 140.2873 | 21.91505 | 9.05296 | 5.78E-04 | 0.05 | S | 68.16306 | 212.4115 |
| 0.02 vs 0.2 | 105.4339 | 21.91505 | 6.80382 | 0.00498 | 0.05 | S | 33.30973 | 177.5581 |
| 0.02 vs 0.1 | 55.09683 | 21.91505 | 3.55549 | 0.16303 | 0.05 | NS | −17.0274 | 127.221 |
| 0.02 vs 0.05 | 51.7058 | 21.91505 | 3.33666 | 0.20378 | 0.05 | NS | −20.4184 | 123.83 |

^{a}The different experimental groups are referred to as “X vs Y”, where X and Y stand for the respective heating rate (° C./min).
^{b}S indicates p < 0.05 (significant); NS indicates p > 0.05 (not significant).

TABLE 7
Complete statistical significance data (p values) of the mineral anisotropy pairwise
comparisons as a function of heating rate (corresponding to FIG. 5f) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 0.2 vs 0.5 | 0.01047 | 0.03599 | 0.41125 | 0.99816 | 0.05 | NS | −0.10799 | 0.12892 |
| 0.1 vs 0.5 | 0.09867 | 0.03599 | 3.87676 | 0.11633 | 0.05 | NS | −0.01979 | 0.21712 |
| 0.1 vs 0.2 | 0.0882 | 0.03599 | 3.46551 | 0.17883 | 0.05 | NS | −0.03026 | 0.20666 |
| 0.05 vs 0.5 | 0.1633 | 0.03599 | 6.4163 | 0.00744 | 0.05 | S | 0.04484 | 0.28176 |
| 0.05 vs 0.2 | 0.15283 | 0.03599 | 6.00505 | 0.0115 | 0.05 | S | 0.03438 | 0.27129 |
| 0.05 vs 0.1 | 0.06463 | 0.03599 | 2.53954 | 0.42599 | 0.05 | NS | −0.05382 | 0.18309 |
| 0.02 vs 0.5 | 0.37287 | 0.03599 | 14.65048 | 8.86E-06 | 0.05 | S | 0.25441 | 0.49132 |
| 0.02 vs 0.2 | 0.3624 | 0.03599 | 14.23923 | 1.15E-05 | 0.05 | S | 0.24394 | 0.48086 |
| 0.02 vs 0.1 | 0.2742 | 0.03599 | 10.77372 | 1.36E-04 | 0.05 | S | 0.15574 | 0.39266 |
| 0.02 vs 0.05 | 0.20957 | 0.03599 | 8.23418 | 0.00122 | 0.05 | S | 0.09111 | 0.32802 |

^{a}The different experimental groups are referred to as “X vs Y”, where X and Y stand for the respective heating rate (° C./min).
^{b}S indicates p < 0.05 (significant); NS indicates p > 0.05 (not significant).

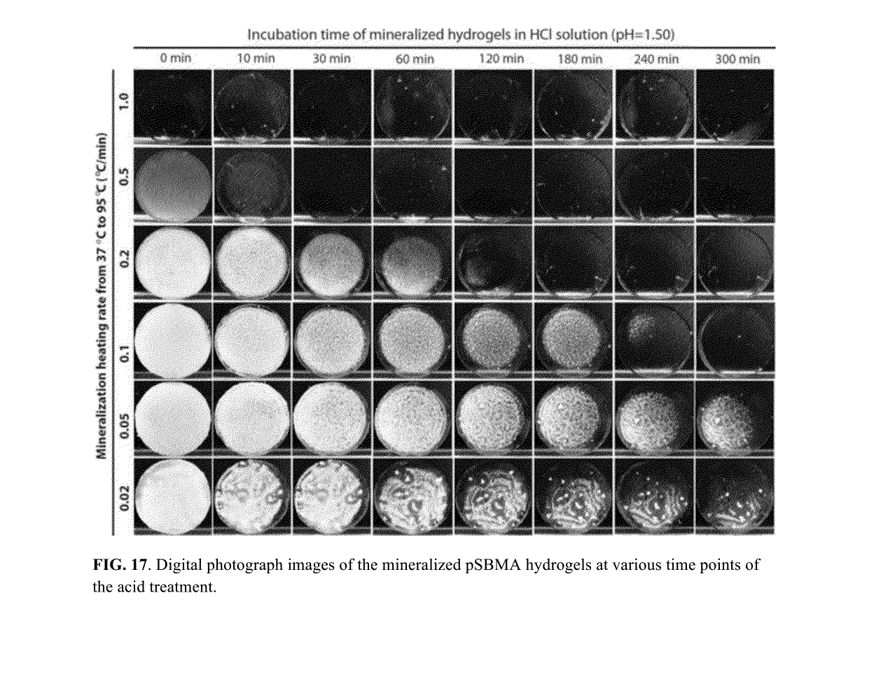

TABLE 8
Complete statistical significance data (p values) of the mechanical property pairwise
comparisons as a function of heating rate (corresponding to FIG. 17b) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 1 vs control | 2.3 | 1.4682 | 2.21543 | 0.70375 | 0.05 | NS | −2.7133 | 7.3133 |
| 0.5 vs control | 6.48 | 1.4682 | 6.24172 | 0.00817 | 0.05 | S | 1.4667 | 11.4933 |
| 0.5 vs 1 | 4.18 | 1.4682 | 4.0263 | 0.13304 | 0.05 | NS | −0.8333 | 9.1933 |
| 0.2 vs control | 10.27667 | 1.4682 | 9.89878 | 1.00E-04 | 0.05 | S | 5.26337 | 15.28997 |
| 0.2 vs 1 | 7.97667 | 1.4682 | 7.68335 | 0.00133 | 0.05 | S | 2.96337 | 12.98997 |
| 0.2 vs 0.5 | 3.79667 | 1.4682 | 3.65706 | 0.20213 | 0.05 | NS | −1.21663 | 8.80997 |
| 0.1 vs control | 14.03333 | 1.4682 | 13.51731 | 2.66E-06 | 0.05 | S | 9.02003 | 19.04663 |
| 0.1 vs 1 | 11.73333 | 1.4682 | 11.30188 | 2.27E-05 | 0.05 | S | 6.72003 | 16.74663 |
| 0.1 vs 0.5 | 7.55333 | 1.4682 | 7.27559 | 0.0022 | 0.05 | S | 2.54003 | 12.56663 |
| 0.1 vs 0.2 | 3.75667 | 1.4682 | 3.61853 | 0.2108 | 0.05 | NS | −1.25663 | 8.76997 |
| 0.05 vs control | 16.14667 | 1.4682 | 15.55293 | 4.24E-07 | 0.05 | S | 11.13337 | 21.15997 |
| 0.05 vs 1 | 13.84667 | 1.4682 | 13.3375 | 3.15E-06 | 0.05 | S | 8.83337 | 18.85997 |
| 0.05 vs 0.5 | 9.66667 | 1.4682 | 9.31121 | 1.93E-04 | 0.05 | S | 4.65337 | 14.67997 |
| 0.05 vs 0.2 | 5.87 | 1.4682 | 5.65415 | 0.01742 | 0.05 | S | 0.8567 | 10.8833 |
| 0.05 vs 0.1 | 2.11333 | 1.4682 | 2.03562 | 0.77316 | 0.05 | NS | −2.89997 | 7.12663 |
| 0.02 vs control | 23.25333 | 1.4682 | 22.39827 | 3.43E-07 | 0.05 | S | 18.24003 | 28.26663 |
| 0.02 vs 1 | 20.95333 | 1.4682 | 20.18285 | 1.18E-07 | 0.05 | S | 15.94003 | 25.96663 |
| 0.02 vs 0.5 | 16.77333 | 1.4682 | 16.15655 | 2.66E-07 | 0.05 | S | 11.76003 | 21.78663 |
| 0.02 vs 0.2 | 12.97667 | 1.4682 | 12.49949 | 6.94E-06 | 0.05 | S | 7.96337 | 17.98997 |
| 0.02 vs 0.1 | 9.22 | 1.4682 | 8.88097 | 3.17E-04 | 0.05 | S | 4.2067 | 14.2333 |
| 0.02 vs 0.05 | 7.10667 | 1.4682 | 6.84534 | 0.00378 | 0.05 | S | 2.09337 | 12.11997 |

^{a}The different experimental groups are referred to as “X vs Y”, where X and Y stand for the respective heating rate (° C./min), while “control” is the unmineralized pSBMA hydrogel.
^{b}S indicates p < 0.05 (significant); NS indicates p > 0.05 (not significant).

TABLE 9
Complete statistical significance data (p values) of the mineral volume pairwise
comparisons as a function of crosslinker content (corresponding to FIG. 6d) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 1.326 vs 2.651 | 0.54723 | 0.39724 | 1.94822 | 0.73894 | 0.05 | NS | −0.78705 | 1.88152 |
| 0.663 vs 2.651 | 0.84683 | 0.39724 | 3.01484 | 0.33398 | 0.05 | NS | −0.48745 | 2.18112 |
| 0.663 vs 1.326 | 0.2996 | 0.39724 | 1.06661 | 0.97033 | 0.05 | NS | −1.03469 | 1.63389 |
| 0.265 vs 2.651 | 2.90927 | 0.39724 | 10.35736 | 1.04E-04 | 0.05 | S | 1.57498 | 4.24355 |
| 0.265 vs 1.326 | 2.36203 | 0.39724 | 8.40914 | 7.39E-04 | 0.05 | S | 1.02775 | 3.69632 |
| 0.265 vs 0.663 | 2.06243 | 0.39724 | 7.34253 | 0.00238 | 0.05 | S | 0.72815 | 3.39672 |
| 0.133 vs 2.651 | 3.9868 | 0.39724 | 14.19352 | 3.99E-06 | 0.05 | S | 2.65251 | 5.32109 |
| 0.133 vs 1.326 | 3.43957 | 0.39724 | 12.2453 | 1.92E-05 | 0.05 | S | 2.10528 | 4.77385 |
| 0.133 vs 0.663 | 3.13997 | 0.39724 | 11.17868 | 4.88E-05 | 0.05 | S | 1.80568 | 4.47425 |
| 0.133 vs 0.265 | 1.07753 | 0.39724 | 3.83616 | 0.14337 | 0.05 | NS | −0.25675 | 2.41182 |
| 0.027 vs 2.651 | 3.59913 | 0.39724 | 12.81338 | 1.20E-05 | 0.05 | S | 2.26485 | 4.93342 |
| 0.027 vs 1.326 | 3.0519 | 0.39724 | 10.86516 | 6.49E-05 | 0.05 | S | 1.71761 | 4.38619 |
| 0.027 vs 0.663 | 2.7523 | 0.39724 | 9.79854 | 1.79E-04 | 0.05 | S | 1.41801 | 4.08659 |
| 0.027 vs 0.265 | 0.68987 | 0.39724 | 2.45601 | 0.5354 | 0.05 | NS | −0.64442 | 2.02415 |
| 0.027 vs 0.133 | −0.38767 | 0.39724 | 1.38014 | 0.91713 | 0.05 | NS | −1.72195 | 0.94662 |

^{a}The different experimental groups are referred to as “X vs Y”, where X and Y stand for the respective crosslinker content (mol %).
^{b}S indicates p < 0.05 (significant); NS indicates p > 0.05 (not significant).

TABLE 10
Complete statistical significance data (p values) of the calcium content pairwise
comparisons as a function of crosslinker content (corresponding to FIG. 6e) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 1.326 vs 2.651 | 0.15893 | 0.05492 | 4.09227 | 0.10756 | 0.05 | NS | −0.02555 | 0.34342 |
| 0.663 vs 2.651 | 0.14113 | 0.05492 | 3.63395 | 0.17872 | 0.05 | NS | −0.04335 | 0.32562 |
| 0.663 vs 1.326 | −0.0178 | 0.05492 | 0.45832 | 0.99939 | 0.05 | NS | −0.20229 | 0.16669 |
| 0.265 vs 2.651 | 0.35167 | 0.05492 | 9.05484 | 3.76E-04 | 0.05 | S | 0.16718 | 0.53615 |
| 0.265 vs 1.326 | 0.19273 | 0.05492 | 4.96257 | 0.03885 | 0.05 | S | 0.00825 | 0.37722 |
| 0.265 vs 0.663 | 0.21053 | 0.05492 | 5.42089 | 0.02248 | 0.05 | S | 0.02605 | 0.39502 |
| 0.133 vs 2.651 | 0.56207 | 0.05492 | 14.47229 | 3.22E-06 | 0.05 | S | 0.37758 | 0.74655 |
| 0.133 vs 1.326 | 0.40313 | 0.05492 | 10.38002 | 1.02E-04 | 0.05 | S | 0.21865 | 0.58762 |
| 0.133 vs 0.663 | 0.42093 | 0.05492 | 10.83834 | 6.65E-05 | 0.05 | S | 0.23645 | 0.60542 |
| 0.133 vs 0.265 | 0.2104 | 0.05492 | 5.41745 | 0.02257 | 0.05 | S | 0.02591 | 0.39489 |
| 0.027 vs 2.651 | 0.92847 | 0.05492 | 23.90649 | 2.39E-07 | 0.05 | S | 0.74398 | 1.11295 |
| 0.027 vs 1.326 | 0.76953 | 0.05492 | 19.81421 | 1.16E-07 | 0.05 | S | 0.58505 | 0.95402 |
| 0.027 vs 0.663 | 0.78733 | 0.05492 | 20.27253 | 2.54E-07 | 0.05 | S | 0.60285 | 0.97182 |
| 0.027 vs 0.265 | 0.5768 | 0.05492 | 14.85165 | 2.42E-06 | 0.05 | S | 0.39231 | 0.76129 |
| 0.027 vs 0.133 | 0.3664 | 0.05492 | 9.4342 | 2.56E-04 | 0.05 | S | 0.18191 | 0.55089 |

^{a}The different experimental groups are referred to as “X vs Y”, where X and Y stand for the respective crosslinker content (mol %).
^{b}S indicates p < 0.05 (significant); NS indicates p > 0.05 (not significant).

When slower heating rates (0.1-0.2° C./min) were employed, larger spherical mineral nodules (Type II, FIG. 5) with narrow Gaussian mineral size distribution (diameters 30-60 μm; FIGS. 6a & 6b, FIGS. 13, 16b & 16f) resulting from more sufficient time for ion diffusions and mineral growth were indeed obtained. The increase in size of the mineral nodules was accompanied by a relative increase in anisotropy (FIG. 6f). At the heating rate of 0.05° C./min, even larger spherical mineral domains containing multiple nucleation centers, with their sizes deviating from the typical Gaussian distributions, were formed (FIGS. 6a & 6b, FIGS. 16c & 16g). Such morphologies represent the departure from the typical type II mineral morphology (FIG. 5). When the heating rate was further reduced to 0.02° C./min, bicontinuous mineral aggregates of small, Liesegang ring-like, spherical nodules were formed in high anisotropy across the hydrogel (FIGS. 6a, 6b & 6f, FIGS. 13, 14, 16d & 16h). (Henisch, et al. 1986 J Cryst Growth 75:203-11.) The relative lower level of supersaturation and the more sufficient time resulting from the slower increase in pH with the much reduced heating rate is expected to allow the slowly grown minerals to aggregate by interparticle forces. (Benning, et al. 2008 Nucleation, growth, and aggregation of mineral phases Mechanisms and kinetic controls. In: Brantley S L, Kubicki J D, aWhite A F, editors. Kinetics of Water-Rock Interaction: Springer; p. 840.) The heating rate-dependent density, volume and spatial distribution of the minerals within the mineralized composites were also confirmed by the temporal changes in appearance of the composites as they were demineralized by acid treatment (FIG. 17).

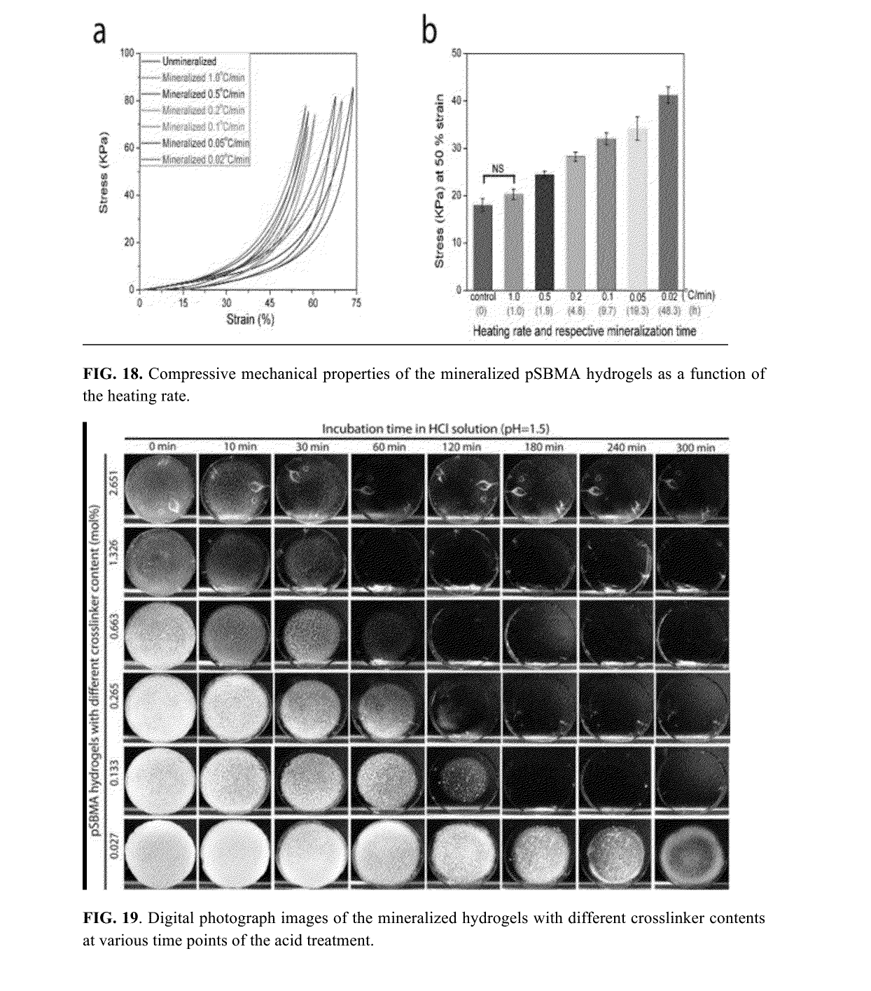

The mineral nodules in all composites examined were structurally well-integrated with the hydrogel matrix, as evidenced by SEM micrographs of the cross-sections of the composites (FIG. 16). No delamination of the spherical mineral nodules from the hydrogel matrix was observed when the air-dried composites were fractured to expose the cross-sections. In addition, observed were steady increases in stiffness and compressive modulus of the hydrated composites as the total mineral contents increased with slower heating rates (FIG. 18), further supporting the good structural integration between the organic and inorganic components. It is likely that the increased connectivity of the mineral domains (higher anisotropy) within the composite mineralized with the slowest heating rate examined (0.02° C./min) also contributed to further improvements in its compressive modulus (e.g. modulus at 50% strain, FIG. 18b) as there was no significant difference between its total mineral content and that mineralized with the heating rate of 0.05° C./min.

Regulating HA Mineralization Outcome by Crosslinking Content of pSBMA

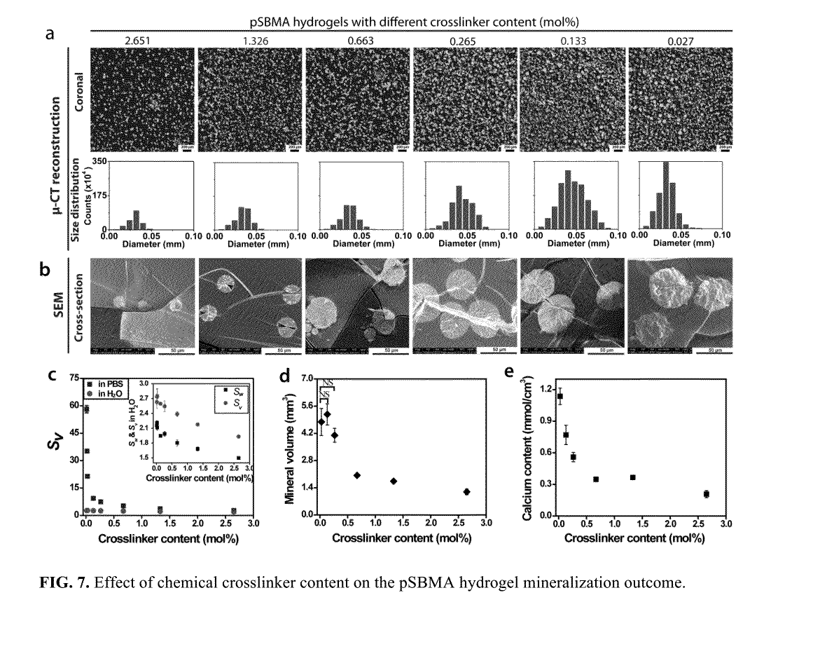

The chemical crosslinker content was found to significantly influence the solute diffusibility as a result of the unique swelling behavior of the zwitterionic hydrogel in a high-salt environment (FIG. 7c) such as the supersaturated mineralization solution, thereby influencing the mineralization outcome. Six hydrogels with EGDMA crosslinker contents ranging from 0.027 to 2.651 mol % were mineralized with a fixed heating rate of 0.2° C./min (Table 2). The mineral volume, mineral size distribution, and total mineral content of these composites, determined by μ-CT analyses and calcium release assays, inversely correlated with the crosslinker content (FIGS. 7a, 7b, 7d & 7e), with substantial mineralization across the 3-D scaffolds (FIG. 7a, FIGS. 19 & 20).

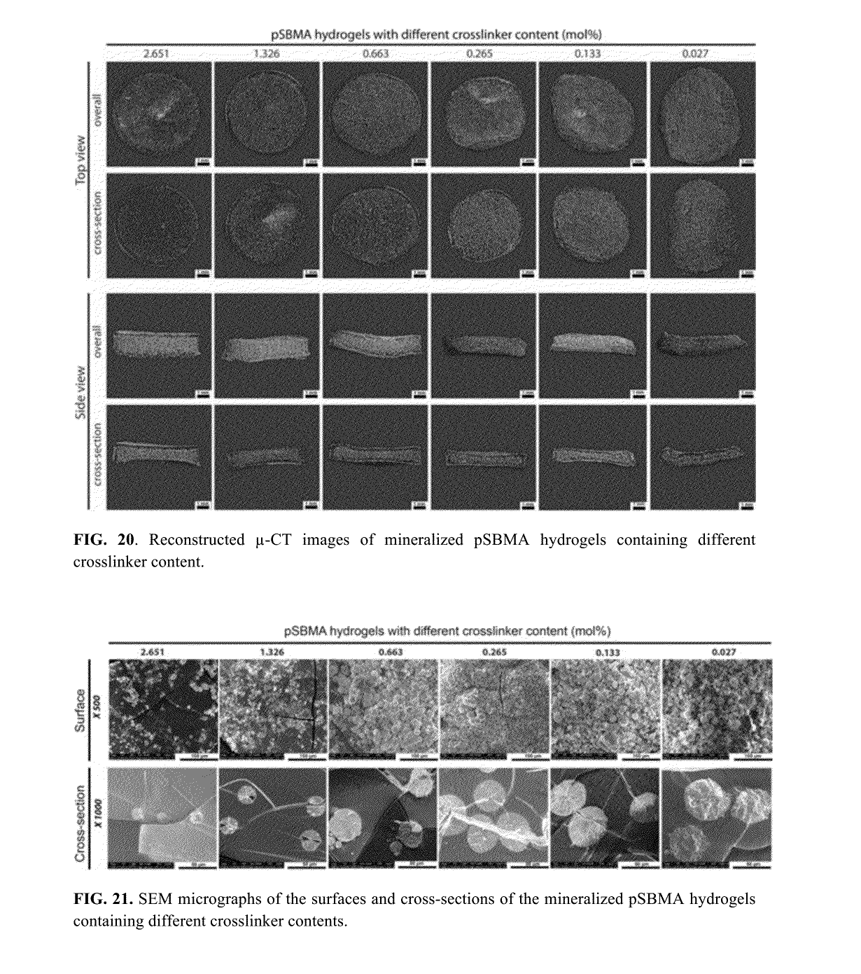

SEM micrographs (FIG. 7b, FIG. 21) revealed that pSBMA with higher crosslinker contents (2.651 and 1.326 mol %) were mineralized with smaller spherical mineral nodules (type I, FIG. 5) while those with medium crosslinker contents (0.663, 0.265, and 0.133 mol %) were mineralized with larger spherical mineral nodules (type II, FIG. 5) within the hydrogel interior. The increase in size of the mineral domains (FIG. 7a) with decreasing crosslinker content at the given heating rate could be attributed to the significantly higher swelling ratio for the hydrogels with lower crosslinker content (e.g. volume swelling ratios in PBS: ˜22 for the 0.027 mol % gel vs. ˜2 for the 2.651 mol % gel, FIG. 7c). The significant increase in volume of the swollen hydrogel may have further facilitated the zwitterionic ligand-mediated active infiltration of precursor ions and more sustained growth of mineral domains.

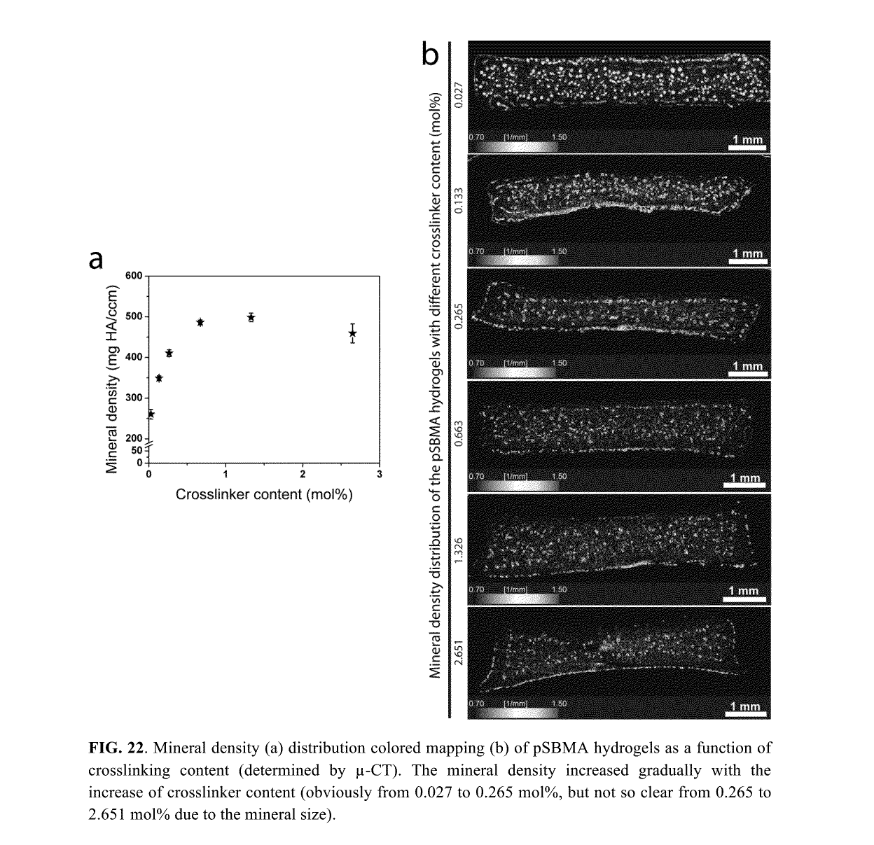

Closer examination of the composites mineralized from the hydrogel with very low crosslinker content (0.027 mol %) by SEM (FIG. 21) revealed large spherical mineral domains comprising of multiple nucleation centers, signaling a departure from the typical type II mineral morphology and the transitioning into the type III mineral morphology where smaller mineral nodules aggregate. Interestingly, unlike mineral volume and total calcium content, the mineral density of these composites steadily increased with increasing crosslinker contents from 0.027 to 0.663 mol %, before reaching a plateau at the crosslinker content beyond 1.326 mol % (FIG. 22, note that no statistically significant difference in mineral density was observed from the composites with 1.326 and 2.651 mol % crosslinker contents). The loosely crosslinked hydrogels have likely allowed better diffusion of precursor ions, thereby replenishing precursor ions due to mineral nucleation and growth in a timely manner to sustain a relative high supersaturation (S) within the hydrogel microenvironment, resulting in a fast mineral growth rate J_{G}. (Mann S. 2001 Biomineralization: Principles and Concepts in Bioinorganic Materials Chemistry. Oxford: Oxford University Press.)

TABLE 11
Complete statistical significance data (p values) of the mineral density pairwise
comparisons as a function of crosslinker content (corresponding to FIG. 22a) as
determined by one-way ANOVA with Tukey's multiple comparison tests.

| Comparison | Mean | | | | | | | |
|---|---|---|---|---|---|---|---|---|
| of Groups^{a} | Diff | SEM | q Value | Prob | Alpha | Sig^{b} | LCL | UCL |
| 1.326 vs 2.651 | 39.262 | 12.30355 | 4.51291 | 0.06614 | 0.05 | NS | −2.06465 | 80.58865 |
| 0.663 vs 2.651 | 27.004 | 12.30355 | 3.10393 | 0.30696 | 0.05 | NS | −14.3227 | 68.33065 |
| 0.663 vs 1.326 | −12.258 | 12.30355 | 1.40898 | 0.9105 | 0.05 | NS | −53.5847 | 29.06865 |
| 0.265 vs 2.651 | −48.6787 | 12.30355 | 5.5953 | 0.01825 | 0.05 | S | −90.0053 | −7.35202 |
| 0.265 vs 1.326 | −87.9407 | 12.30355 | 10.10821 | 1.32E-04 | 0.05 | S | −129.267 | −46.614 |
| 0.265 vs 0.663 | −75.6827 | 12.30355 | 8.69923 | 5.44E-04 | 0.05 | S | −117.009 | −34.356 |
| 0.133 vs 2.651 | −110.069 | 12.30355 | 12.65176 | 1.37E-05 | 0.05 | S | −151.396 | −68.7427 |
| 0.133 vs 1.326 | −149.331 | 12.30355 | 17.16467 | 4.24E-07 | 0.05 | S | −190.658 | −108.005 |
| 0.133 vs 0.663 | −137.073 | 12.30355 | 15.75569 | 1.23E-06 | 0.05 | S | −178.4 | −95.7467 |
| 0.133 vs 0.265 | −61.3907 | 12.30355 | 7.05646 | 0.00329 | 0.05 | S | −102.717 | −20.064 |
| 0.027 vs 2.651 | −195.468 | 12.30355 | 22.46778 | 9.89E-07 | 0.05 | S | −236.795 | −154.141 |
| 0.027 vs 1.326 | −234.73 | 12.30355 | 26.98069 | 0 | 0.05 | S | −276.057 | −193.403 |
| 0.027 vs 0.663 | −222.472 | 12.30355 | 25.57171 | 9.85E-08 | 0.05 | S | −263.799 | −181.145 |
| 0.027 vs 0.265 | −146.789 | 12.30355 | 16.87248 | 5.32E-07 | 0.05 | S | −188.116 | −105.463 |
| 0.027 vs 0.133 | −85.3987 | 12.30355 | 9.81602 | 1.76E-04 | 0.05 | S | −126.725 | −44.072 |

^{a}The different experimental groups are referred to as “X vs Y”, where X and Y stand for the respective crosslinker content (mol %).

### A Model of Synergistic Modulation of Mineralization Outcome by Heating Rates and Crosslinker Contents

The above investigation of the roles of heating rate (mineralization time) and covalent crosslinker content on the mineralization outcome of the zwitterionic pSBMA hydrogel supported that three predominant types of mineral domains can be obtained by tuning these experimental parameters (FIG. 5). It was found that lower contents of isolated small spherical mineral nodules (Type I) tended to form with either faster heating rates (1.0 and 0.5° C./min) or higher pSBMA crosslinker contents (2.651 and 1.326 mol %). These conditions limited either the time for sufficient precursor ion diffusions across the hydrogel and/or space for continued mineral growth within the hydrogel. Higher mineral content and larger spherical mineral nodules within the pSBMA hydrogels (type II) could be obtained with medium heating rates (0.2 and 0.1° C./min) or medium crosslinking densities (0.663, 0.265 and 0.133 mol %) as these conditions allowed more sufficient time and space for sufficient ion diffusion, maintaining the local supersaturation for sustained mineral growth. Formation of stripe-like bicontinuous mineral aggregates of small spherical mineral nodules within the hydrogel (Type III) could be accomplished with either much slower heating rate (0.02° C./min) or very low crosslinking densities (<0.027 mol %). These conditions may have allowed sufficient time, space or polymer chain mobility (e.g. higher polymer chain motilities are expected within the less crosslinked hydrogel network) for the slow-growing mineral domains to interact and aggregate. Borderline crosslinker content such as 0.027 mol % and borderline heating rate such as 0.05° C./min led to mineral morphologies departing from Type II and transitioning to Type III.

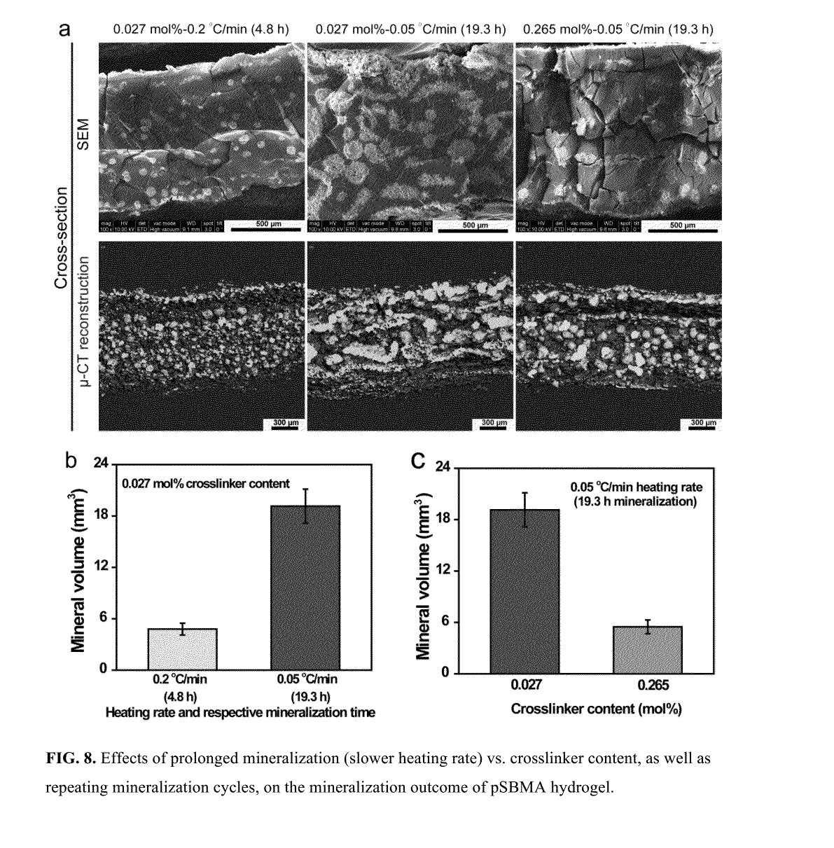

To validate this proposed model, it was demonstrated that lowering the mineralization heating rate of the pSBMA hydrogel with borderline crosslinker content of 0.027 mol % from 0.2 to 0.05° C./min (increasing mineralization time from 4.8 to 19.3 h) indeed resulted in the transitioning from Type II spherical mineral morphology to Type III stripe-like morphology with increasing mineral volume (FIGS. 8a & 8b). Likewise, lowering the crosslinker content from a medium 0.265 mol % to the lower 0.027 mol % at a given borderline heating rate of 0.05° C./min also led to the transitioning from the Type II morphology to the higher mineral content, stripe-like type III morphologies (FIGS. 8a & 8c).

### Effect of Repeated Mineralization Cycles of Mineralization Outcome

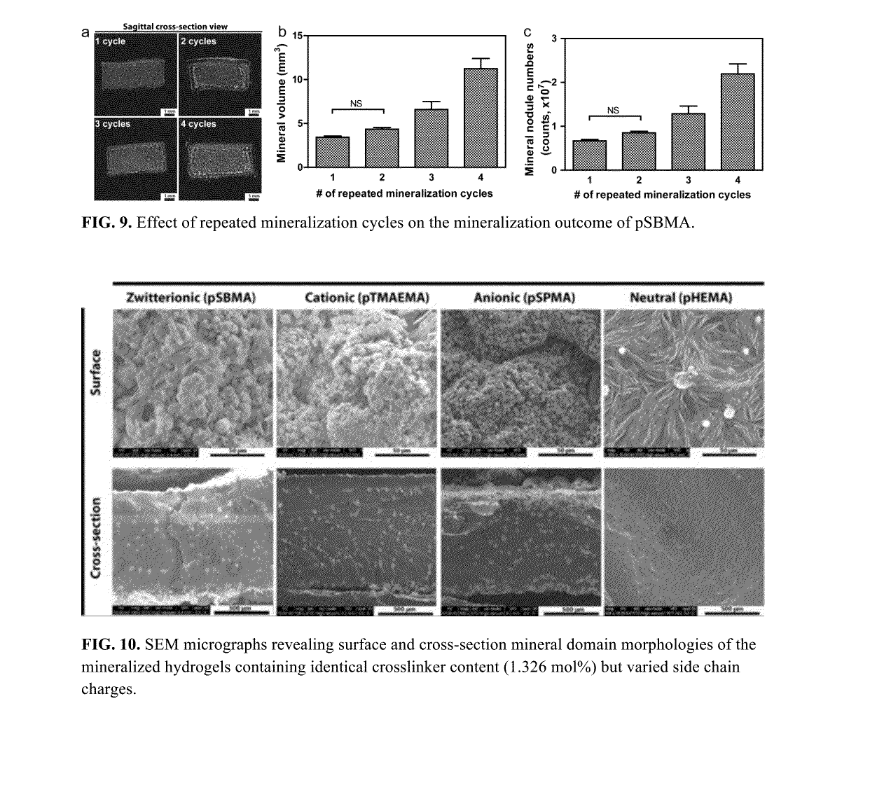

Furthermore, also demonstrated was that by repeating the urea thermal decomposition-mediated HA-mineralization process to pSBMA (e.g. with the borderline 0.027 mol % crosslinker content and a heating rate of 0.2° C./min), the overall mineral content and the number of the mineral modules were steady increased (FIG. 9). This finding offers a practical approach to further modulating the mineralization outcome, achieving even more extensively mineralized zwitterionic hydrogel-HA composites if desired.

### EXAMPLES

### Preparation of Chemically Crosslinked Hydrogels

Four types of hydrogels, zwitterionic poly[2-(methacryloyloxy)ethyl]dimethyl-(3-sulfopropyl)ammonium hydroxide (pSBMA), cationic poly[2-(methacryloyloxy)ethyl]trimethylammonium (pTMAEMA), anioic poly(3-sulfopropyl methyacrylate) (pSPMA), and uncharged poly(2-hydroxyethyl methacrylate) (pHEMA), were prepared (Table 1, No. 1-4). Monomers SBMA, TMAEMA, SPMA and HEMA and crosslinker ethylene glycol dimethacrylate (EGDMA) were purchased from Aldrich (St. Louis, Mo.), with the radical inhibitors being removed by distillation (for HEMA), aluminum oxide column followed by freeze-drying (for PTMAEMA) or molecular sieves (for EGDMA) prior to use. In a typical procedure, 2 mmol respective monomer was combined with 50 μL of ethanol solution of EGDMA (10%, v/v), 100 μL PBS solution of 2,2′-Azobis[2-methyl-N-(2-hydroxyethyl)propionamide] (VA-086, 2%, w/v), and 1850 μL of Milli-Q water. The mixture was bath-sonicated to a clear solution and transferred to a custom-made Teflon mold with cylindrical (6 mm in diameter) and square prism (5 mm×5 mm) wells (50 μL/well or 100 μL/well) and solidified under irradiation (365 nm, 10 min). The crosslinked hydrogels were equilibrated in Milli-Q water for 2 days at 37° C. with exchange of water every 12 h to ensure the removal of residue initiators or any unreacted monomers. The hydrogels were stored in Milli-Q water until mineralization or further characterizations.

To further study the effect of crosslinker contents on the HA-mineralization outcome of zwitterionic hydrogels, eight pSBMA hydrogels with different crosslinker contents, 2.651 mol % (#1), 1.326 mol % (#2), 0.663 mol % (#3), 0.265 mol % (#4), 0.133 mol % (#5), 0.027 mol % (#6), 0.020 mol % (#7), and 0.013 mol % (#8), as detailed in Table 2, were also prepared using the procedure described above.

### Swelling Ratios of the Hydrogel

The swelling ratios of the hydrogel by weight (S_{w}) and by volume (Sᵥ) were determined in Milli-Q water or in PBS (pH=7.4) at room temperature according to Equations 4 and 5:

$${S}_{w} = \frac{{W}_{h} - {W}_{d}}{{W}_{d}} \qquad Eq \text{-} 4$$

where Wₕ and W_{d} are the weight of a hydrogel in fully hydrated state and dry state, respectively.

$${S}_{v} = \frac{{V}_{h} - {V}_{d}}{{V}_{d}} \qquad Eq \text{-} 5$$

where Vₕ and V_{d} are the volume of a hydrogel in fully hydrated state and dry state, respectively.

### Mineralization of the Hydrogel

Mineralization was carried out by controlled heating of the hydrogels in a urea-containing, acidic solution of hydroxyapatite from 37° C. to 95° C. using a protocol modified over a previous report. (Song, et al. 2003 J Am Chem Soc 125:1236-43.) Mineralization stock solution was prepared by suspending hydroxyapatite (7.37 g, 34-40% Calcium content, Alfa Aesar) in 500-mL aqueous solution of urea (2 M), followed by the addition of concentrated hydrochloric acid under constant stirring until a clear soluble solution was obtained (final pH 2.5-3.0). Six to ten hydrogels were placed in an Erlenmeyer flask filled with 30 mL of mineralization solution and covered with a perforated aluminum foil. The flask was placed in a high-temperature silicone oil bath with the mineralization solution completely submerged under the oil and heated using a 100-watt immersion heater (Glo-Quartz LHP-IAH4) equipped with a programmable temperature controller (Eurotherm 2408). Controlled heating from 37° C. to 95° C. was carried out at a heating rate of 0.2° C./min. Mineralized hydrogels were bath-sonicated for 5 min in Milli-Q water to ensure removal of loosely bound minerals, followed by further incubation in fresh Milli-Q water at 37° C. for two days with regular water change of every 12 h to ensure removal of the residual mineralization precursor ions trapped within the mineralized hydrogel. All mineralized specimens were able to recover from their swollen state in high salt mineralization solution to similar dimensions in water at the end of the 2-day post-mineralization equilibration.

To examine the effect of repeated mineralization, pSBMA hydrogels with a crosslinker content of 0.265 mol % were repeatedly mineralized in fresh mineralization solution at a heating rate of 0.2° C./min up to four times.

Temporal pH Changes of the Mineralization Solution with Different Heating Rates

To monitor the changes of pH of the mineralization solution over time with different heating rates, six groups of Erlenmeyer flasks (three flasks per group with each flask containing 45 mL of mineralization solution and one pSBMA hydrogel with a crosslinker content of 0.265 mol %) were covered with perforated aluminum foils and heated from 37° C. to 95° C. at a heating rate of 0.02 0.05, 0.1, 0.2, or 1.0° C./min, respectively. At pre-determined sampling intervals, 2 mL of mineralization solution was retrieved from the flask, cooled to rt before its pH was measured by a VWR Symphony pH meter. Averaged pH values along with the standard deviations for each of the 15 time points at each heating rate were plotted over mineralization time.

### Quantitation of Calcium Content of Mineralized Hydrogels

Total calcium content as a function of mineralization conditions was determined by quantifying the Ca²⁺ ions released from each mineralized hydrogel in a hydrochloric acid solution with a Thermo Scientific calcium ion selective electrode attached to a VWR Symphony pH/ISE meter. In a typical procedure, the mineralized hydrogel was placed in 10 mL of hydrochloric acid solution (pH 3) in a 20-mL glass vial and the pH was adjusted by concentrated hydrochloric acid to around 2.1. The mineral was allowed to be fully released from the hydrogel under constant shaking of the acidic solution on an orbital shaker, as indicated by reaching a transparent appearance of the gel. Ionic Strength Adjustment buffer (ISA, 4 M KCl solution, VWR, 200 μL) was added to the acidic solution containing the released calcium prior to measurement by the calcium ion selective electrode. The total calcium content of each type of mineralized hydrogel (N=3) was determined using a standard curve generated by a series of acidic (pH 2.1) aqueous Ca²⁺ ion standard solutions containing 0.1, 0.01, 0.001, and 0.0001 M CaCl₂.

Real-Time Monitoring of the Mineral Release from the Hydrogels

The progress of the mineral release from the mineralized hydrogel in hydrochloric acid (pH 1.5, 4.5 mL per specimen) was monitored by digital photographs of the specimen taken at 0, 10, 30, 60, 120, 180, 240, and 300 min of the incubation using a digital camera (Canon, VIXIA HF200).

### Zeta Potential Measurement

To examine how the ionic status of the zwitteironic pSBMA varied as a function of significant pH changes during the mineralization, the zeta potentials of linear pSBMA polymer (synthesized in our lab using the well-controlled atom transfer radical polymerization process, Mn=12424, PDI=1.09) under varied solution pH were measured on a Zetasizer (Zen3600, 633 nm laser, Malvern). Linear pSBMA solutions (3 mg/mL in 0.015 M NaCl aq. solution) were adjusted with a final pH of 3.01, 3.99, 5.30, 6.45, 7.03, 7.43 or 9.17 by aqueous HCl or NaOH, and placed in disposable capillary cells (DTS1061, Malvern) for zeta potential measurement at 25° C.

### Mechanical Testing

It is known that increasing mineral content in a poorly integrated polymer-mineral composite can result in the deterioration of the mechanical properties of the composite. Thus, it is important to examine the macroscopic mechanical properties of the mineralized pSBMA hydrogels under the context of increasing mineral contents. The compressive behavior of fully hydrated mineralized zwitterionic hydrogel (#4 in Table 2, with a crosslinker content of 0.265 mol %) as a function of the heating rate, along with the un-mineralized control, was evaluated on a Q800 dynamic mechanical analyzer (DMA, TA Instruments) equipped with a submersion compression fixture. The instrument has an 18-N load cell, a force resolution of 10 μN and a displacement resolution of 1.0 nm. Each specimen fully equilibrated in water was compressed in a force-controlled mode at 25° C., ramping from 0.02 to 6.0 N at a rate of 1.0 N/min then back to 0.02 N at the same rate. A pre-load of 0.02 N was applied. The averaged compressive stresses at 50% strain (N=3) of the composites were plotted as a function of the heating rate.

### Microcomputed Tomography (μ-CT)

Mineralized hydrogel specimens (N=3) were scanned on a Scanco m-CT40 scanner. The effective voxel size of the reconstructed images was 8×8×8 mm³ Data were globally thresholded and reconstructed to quantify the mineral volume (MV, mm³), mineral density (MD, mg HA/ccm), mineral size (diameter or thickness, mm) distribution, mineral density (1/mm) distribution and anisotropy of the composites. MV and MD were determined by a direct analysis. MS/MV and anisotropy were determined by a TRI (plate mode) analysis. Direct thickness distributions of the 3-D mineralized structures were determined with a volume-based thickness definition. It defines the local thickness at a given point in the mineralized structure as the diameter of the largest sphere including the point that can be fitted within the structure. Mineral density (1/mm) distributions within the hydrogels were visualized by reconstructing the respective AIM file with a colored density gradient range of 0.7-1.5 (1/mm) An un-mineralized hydrogel was also scanned as a negative control to ensure proper setting of the threshold for analyses.

### Cell Encapsulation in Hydrogels

The cyto-compatibility of three types of hydrogels with different overall charges, zwitterionic pSBMA, cationic pTMAEMA, and anioic pSPMA were investigated by the viability of rat bone marrow stromal cells (rMSCs) encapsulated in these hydrogels. In a typical procedure, 2 mmol of the respective hydrogel monomer was dissolved in 2 mL of expansion medium (a-MEM supplemented with 20% FBS, 1% penicillin, 1% streptomycin, and 2% glutamine) mixed with 100 μL PBS solution of 2 wt % VA-086 (Wako) and a pre-determined amount of water-soluble crosslinker polyethylene glycol dimethacrylate (PEGDMA, Mₙ=750, Sigma-Aldrich) as described listed in Table 1 (No. 5-7). Given the drastically different swelling behavior of cationic, anionic and zwitterionic hydrogels, the PEGDMA crosslinker contents applied to the respective hydrogels were adjusted to achieve comparable equilibrium gel volumes in culture media, thus comparable initial cell encapsulation density within the various hydrogels. The mixture was sterile-filtered through a 0.2-μm sterile polyethersulfone syringe filter. Passage 1 rMSC, isolated from the femoral marrow canal of skeletally mature SASCO SD rats (Charles River Lab, 8-10 week old) and enriched by adherent culture, were plated in expansion media for 24 h before they were trypsinized, counted, and suspended into the respective monomer/crosslinker/initiator mixture solutions (10⁶ cells/mL). (Xu, et al. 2011 Chem-Asian J. 6:2730-7.) The rMSC-containing solution was then transferred to a custom-made Teflon mold of square prism wells (5 mm×5 mm; 50 μL/well) and solidified under the irradiation of 365-nm light for 10 min in a sterile hood. All cell-hydrogel constructs were cultured for up to 4 days in humidified incubation (5% CO₂, 37° C.) before being subjected to live/dead cell staining.

### Live/Dead Cell Staining and Confocal Microscopy

The hydrogel-cell constructs were stained using a LIVE/DEAD viability/cytotoxicity kit (Molecular Probes) following the vendor's protocol. Live cells were stained with green fluorescence by intracellular esterase-catalyzed hydrolysis of Calcein AM, and dead cells were stained red by ethidium homodimer-1 penetrated through the damaged membranes and bound with nucleic acids. After being stained for 30-45 min at 37° C., the stained hydrogel-cell construct was retrieved and mounted in a Cellview culture dish (Greiner) and imaged on a Leica TCS SP2 confocal microscope. Calcein was excited at 488 nm and observed with the FITC filter (518-542 nm) while ethidium homodimer-1 was excited at 543 nm and observed with the TEXAS RED filter (625-665 nm). Confocal Z-stack images of encapsulated rMSC over the depth of 80 μm (8 consecutive 10 μm slices) were overlaid.

### MTT Cell Viability Assay

The viability of the rMSCs encapsulated in the zwitterionic pSBMA and the uncharged PEGMA control with established cytocompatibility (prepared by crosslinking PEGMA monomer from Sigma-Aldrich, Mn=360, at the same crosslinker content of 1.33 mol % applied to pSBMA) were evaluated by an MTT cell proliferation kit (Roche) at 24 and 96 h after the initial cell encapsulation. The gel-cell constructs were cultured in expansion media (a-MEM supplemented with 20% FBS, 1% penicillin, 1% streptomycin, and 2% glutamine). MTT labeling reagent (50 μL) was added to each well containing culture media & cell-hydrogel construct and incubated for 11 h in humidified incubation (5% CO₂, 37° C.). Solubilization solution (500 μL) was then added and incubated for an additional 12 h to release the purple formazan from the 3-D hydrogels. The absorbance of the collected supernatant (N=3) was read at 570 nm (with background correction read at 690 nm) on a MULTISCANFC spectrophotometer (Thermo Scientific).

### Scanning Electron Microscopy (SEM)

The mineral morphologies at both the surface and the cross-section of air-dried mineralized hydrogels, sputter coated with 4-nm gold, were observed by SEM on a Quanta 200 FEG MKII microscope equipped with an Oxford-Link Inca 350 X-ray spectrometer (Oxford Instruments) under an accelerating voltage of 10 kV and a spot size of 3.0 μm. For the cross-section observation, air-dried mineralized hydrogels were bisected by a razor blade.

### Focused Ion Beam (FIB) Processing and Transmission Electron Microscopy (TEM)

Air-dried cubic mineralized specimens were first bisected with a razor blade to expose the mineral domains at the cross-section and sputter coated with 8 nm of gold. Selected mineral domains at the cross-section were further sectioned with a Zeiss NVision 40 dual-beam Focused Ion Beam (FIB) using 30 kV Ga⁺ ions. A U-cut (1.5 nA) was performed, followed by further ion polishing (5 kV, 40 pA) to thin the regions of interest to achieve TEM transparency (<100 nm in thickness). The thinned FIB specimens were then examined on an FEI transmission electron microscope (Tecnai 12 Spirit) at 80kV and on a high-resolution transmission electron microscope (JEOL 2000FX) at 200 kV. The relative intensities of the 002, 004, and 211 diffraction arcs or spots in selected area electron diffraction (SAED) were analyzed by ImageJ. The chemical compositions of the thinned mineral specimens (FIB samples) were examined on a JEOL 2010 TEM/STEM equipped with Energy Dispersive X-ray Spectroscopy (EDS). Three random points on each specimen were scanned.

In Vivo Bone Formation Guided by the pSBMA Hydrogel in the Absence of Any Therapeutic Agents Using a 5-mm Rat Femoral Segmental Defect Model

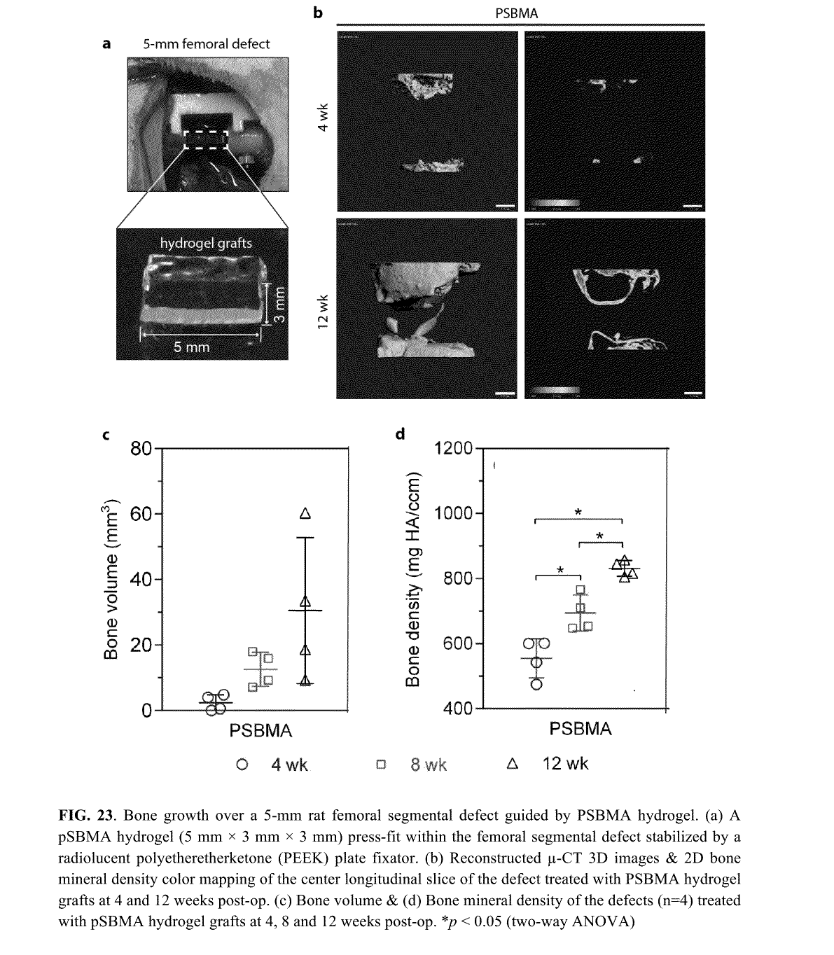

The efficacy of the zwitterionic pSBMA hydrogel (crosslinked with 1.33 mol % PEGDMA) was examined in templating in vivo osteointegration for the repair of 5-mm rat femoral segmental defect (FIG. 23a). Briefly, male Charles River SASCO-SD rats (289-300 g) were sedated and maintained by 2% isoflurane-oxygen throughout the surgery. The mid-shaft of a femur was exposed by a combination of sharp and blunt dissections and the periosteum of the exposed femur was circumferentially removed to emulate a challenging clinical scenario where this important source of progenitor cells and signaling molecules is lost. A radiolucent, weight bearing polyetheretherketone (PEEK) internal fixation plate was secured to the exposed femur with four bicortical screws into predrilled holes. A 5-mm mid-diaphyseal defect was then created using an oscillating Hall saw with parallel blades. The defect site was thoroughly irrigated with saline to remove bone debris and residue of detached periosteum before it was fit with a hydrogel graft without any cells or therapeutic agents. The muscle and skin were closed with resorbable sutures and the rats were given cefazolin (20 mg/kg, once a day) and bupenorphine (0.08 mg/kg, 3 times a day) injections subcutaneously over the next 2 days. Rats were radiographed biweekly post-op to ensure proper graft positioning, and subjected to monthly longitudinal microCT (μ-CT) scans (n=4) to quantitatively monitor the mineralized callus formation until time of sacrifice at 12 weeks post-op. The pSBMA was shown led to steady growth of bony callus over 12 weeks (FIG. 23b), characterized with increasing bony callus volume (FIG. 23c) and bone mineral density (FIG. 23d).

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A composite material comprising 3-dimensioanl network of a synthetic polymer comprising a zwitterionic moiety and one or more structurally integrated mineral component throughout the 3-D network.

2. The composite material of claim 1, wherein the mineral component comprises one or more of calcium apatites, hydroxyapatite, substituted hydroxyapatites, calcium deficient hydroxyapatite, calcium phosphates, octacalcium phosphate, tricalcium phosphate, any transitional mineral phases between amorphous calcium phosphate to crystalline calcium apatite, and amorphous and crystalline forms of calcium carbonate.

3-4. (canceled)

5. The composite material of claim 1, wherein the composite material has isolated or continuous aggregates of spherical mineral nodules having individual mineral nodules sizes from about 1 mm to about 300 mm in diameter.

6. (canceled)

7. The composite material of claim 1, further comprising encapsulated live cells.

8-10. (canceled)

11. The composite material of claim 1, wherein the 3-dimensional polymer network is crosslinked.

12. (canceled)

13. The composite material of claim 1, wherein the 3-dimensional polymer network comprises a zwitterionic moiety.

14. (canceled)

15. The composite material of claim 1, wherein the composite material is cytocompatible and/or biodegradable.

16. (canceled)

17. A method for mediating 3-dimensional mineralization, comprising
providing a mixture comprising:
(a) a 3-dimensional scaffold of a polymer network, wherein the polymer network comprises a zwitterionic moiety;
(b) a mineral precursor; and
(c) a pH mediator;
causing the pH mediator to gradually adjust the pH level of the mixture; and
transforming the mineral precursor into mineral deposits as templated the 3-dimensinal scaffold.

18. The method of claim 17, wherein causing the pH mediator to gradually adjust the pH level of the mixture is by decomposing or evaporating the pH mediator in aqueous solution to generate Off or H.

19. The method of claim 18, wherein causing the pH mediator to gradually adjust the pH level of the mixture is by decomposing or evaporating the pH mediator in aqueous solution to generate OH⁻.

20. The method of claim 17, wherein the pH mediator is selected from urea, ammonium hydroxide, ammonia.

21-22. (canceled)

23. The method of claim 17, wherein the mineral precursor comprises one or more selected from calcium apatites, calcium phosphates, hydroxyapatite, and substituted hydroxyapatites.

24-26. (canceled)

27. The method of claim 17, wherein the polymer network comprises a zwitterionic moiety.

28. (canceled)

29. A 3-dimensional mineralized synthetic scaffold prepared according Claim 17.

30-32. (canceled)

33. An implant comprising a composite material characterized by a 3-dimensional scaffold of a polymer network substantially mineralized therein hydroxyapatite crystals, wherein the polymer comprises a zwitterionic moiety.

34. The implant of claim 33, wherein the composite material is cytocompatible and/or biodegradable.

35. (canceled)

36. The implant of claim 33, being an implant of dental, bone, cartilage, tendon, ligament, osteochondral replacement.

37. The implant of claim 33, further comprising encapsulated live cells.

38-39. (canceled)

40. The implant of claim 33, wherein the composite material has a morphology characterized by a mineral content from about 0.1 wt % to about 95 wt % with isolated or continuous aggregates of spherical mineral nodules with individual mineral nodule sizes from about 1 mm to about 300 mm in diameter.

41. A 3-dimensional scaffold comprising a 3-dimensioanl polymer network, wherein the polymer network comprises zwitterionic moiety, to be utilized as mineralization templates.

42-50. (canceled)
